(19) United States
(12) Patent Application Publication
(10) Pub. No.: US 2022/0226637 A1
(43) Pub. Date: Jul. 21, 2022

(54) INTELLIGENT VECTOR ELECTRODE FOR A PACEMAKER OR AN IMPLANTABLE CARDIOVERTER-DEFIBRILLATOR

(71) Applicant: University Health Network, Toronto (CA)

(72) Inventor: Kumaraswamy Nanthakumar, Mississauga (CA)

(21) Appl. No.: 17/607,600

(22) PCT Filed: May 6, 2020
(86) PCT No.: PCT/CA2020/050620

§ 371 Date: Oct. 29, 2021

Related U.S. Application Data
(60) Provisional application No. 62/843,728, filed on May 6, 2019.

(51) Int. Cl. A61N 1/05; A61N 1/365; A61N 1/37

(52) U.S. Cl. CPC A61N 1/056; A61N 1/371; A61N 1/365

(57) ABSTRACT

A multi-electrode implantable device for sensing cardiac signals and various methods for using the sensed cardiac signals are described herein. The multi-electrode device comprises a tetrahedral electrode cluster at a tip at a distal end of the lead/device; four electrodes embedded in the tetrahedral configuration; and four individual wires extending from the electrodes within the lead for receiving voltages sensed by the four electrodes. The methods can be used for deriving various physiological features that can be used in various ways including: diagnosing a physiological condition, efficient sensing of physiological signals, applying more efficient pacing by a pacemaker and indirect cardiac mapping. One or more of the physiological features may be used for applying appropriate treatment methods by a pacemaker/ICD or for applying cardiac ablation or cryofreezing.

CROSS-REFERENCE

This application claims the benefit of U.S. Provisional Patent Application No. 62/843,728, filed May 6, 2019, and the entire contents of U.S. Provisional Patent Application No. 62/843,728 is hereby incorporated by reference.

### FIELD

Various embodiments are described herein that generally relate to multi-electrodes on a lead for a pacemaker, a leadless pacemaker, or an implantable cardioverter-defibrillator, as well as the methods of using electrical data measured using the electrode for analysis, including sensing, pacing diagnostics, and arrhythmia detection and treatment from implantable devices.

### BACKGROUND

The following paragraphs are provided by way of background to the present disclosure. They are not, however, an admission that anything discussed therein is prior art or part of the knowledge of persons skilled in the art.

Pacemakers and implantable cardioverter-defibrillators (ICD) have electrodes that may be used to sense electrical activity in the heart by measuring a potential difference across two of the electrodes. The electrodes may be located at the tip of the leads. The amount of voltage detected by both the pacemaker and the defibrillator, termed as the “sensing voltage”, is dependent upon the design and placement of the electrodes and are useful for accurately detecting the heart's activity and differentiating from noise. The sensing function of the electrodes is also useful for the adequate function of both the pacemaker and the defibrillator, such as, for example, in facilitating the appropriate timing and delivery of electrical signals to the heart to regulate the electrical activity in the heart. This is additionally useful in a new and evolving field of His pacing, where differentiating atrial signals, His bundle signals, and ventricular signals is difficult, and additionally in leadless pacing, where repositioning of the whole pacemaker is needed, which can produce complications.

When voltage detection is performed between two electrodes of a pacemaker/ICD lead, it is only possible to detect a voltage difference in the orientation in which the electrode is situated. If indeed the lead were to move and/or be displaced, the sensing voltage may likely drop, requiring the repositioning of these leads for better detection of electrical signals. There may then be a requirement for a surgical operation and direct manipulation of the lead or, in the case of leadless pacemakers device, repositioning, which requires an additional operation, as well as the extra cost and risks of a new operation to the patient, including increased risk of infection.

Furthermore, these leads are used to detect changes in rhythm by measuring signals and observing the change in signal morphology. However, these signals are based on voltage differences across two poles, and the sensitivity of the signal change is suboptimal in detecting changes in rhythm. This is a challenge when trying to detect a life-threatening arrhythmia versus a non-life-threatening arrhythmia when the patient is seen in the clinic.

Current day devices cannot detect the direction from which an activation wavefront came and passed by a particular lead. Additionally, when having to perform an ablation, Medical Doctors (MDs) have to determine the area of the heart from which the abnormal pulse originated. If these rhythms were to be recorded by ICD devices, such that the implanted leads have sufficient recording fidelity to determine when and where an electrical cardiac pulse originated from various regions of the heart, then these leads may be used as a tool for mapping the heart while providing stimulus pacing to recreate the exact match, thus identifying the region where the abnormal rhythm originated. The electrical signals from these leads may be used to provide a tool map, which can be used to identify any regions of the heart in need of treatment. Such a tool map may be used to enable a pacing-based detection of at least one arrhythmogenic focus. Bipolar voltages measured from current pacemaker/ICD leads are incapable of providing such mapping information with accuracy.

Current day pacemaker/ICD units cannot determine conduction velocity (CV), which may be valuable in determining drug efficacy and changes in myocardial health status. Current day pacemaker/ICD units cannot determine CV because they are unable to map wavefronts.

### SUMMARY OF VARIOUS EMBODIMENTS

Various embodiments of a lead for a pacemaker or an implantable cardioverter-defibrillator, methods of use thereof, and systems for use therewith, are provided according to the teachings herein.

According to one broad aspect in accordance with the teachings herein, there is provide a multi-electrode implantable device comprising: a lead; a tip at a distal end of the lead; four electrodes embedded in a tetrahedral configuration at the distal end of the lead; and four individual wires extending from the electrodes within the lead for receiving voltages sensed by the four electrodes.

In at least one embodiment, a central electrode of the four electrodes is at the tip of the device, aligning a tip of the tetrahedral configuration with a longitudinal center axis of the lead.

In at least one embodiment, three of the four electrodes are positioned in an equilateral triangular planar configuration to form a base of the tetrahedral configuration near a circumference of the lead, the three electrodes being equally spaced in relation to the central electrode at the distal tip.

In at least one embodiment, the device further comprises a shaft made of soft material encompassing the lead.

In at least one embodiment, the device comprises a single connector pin at a proximal end of the lead that is coupled to the wires and configured to transmit electrophysiological signals sensed by the electrodes to an external device.

In at least one embodiment, the single connector pin is an industry standard pin.

In at least one embodiment, the device comprises a communication unit that is coupled to the four individual leads for wirelessly transmitting the sensed voltages to another device.

In at least one embodiment, the device comprises a miniaturized pacemaker/ICD unit that is coupled to the four individual leads for receiving and processing the sensed voltages.

In another broad aspect, in accordance with the teachings herein there is provided a method of analyzing electrophysiological (EP) data from a 3D multi-electrode device having four electrodes positioned at a distal end of the device in a 3D tetrahedral configuration and the device being located at a heart, the method comprising: sensing unipolar voltages with the four electrodes individually to provide sensed signals; recording the sensed signals as the EP data after signal capture occurs; determining an electric field from the EP data measured by the four electrodes; generating one or more features derived from the electric field; and analyzing the one or more features to determine when a heart rhythm of the heart has an irregular change, is erratic or is abnormal.

In at least one embodiment, the method comprises determining an electric field span potential (EFSP), a geometric profile of the electric field, a travelling wave conduction direction and/or a conduction wave velocity as the one or more features derived from the electric field.

In at least one embodiment, the method comprises determining the EFSP by determining a largest Euclidean distance formed with the electric field and scaling the Euclidean distance with an inter-electrode distance to express the EFSP as a voltage.

In at least one embodiment, the method comprises obtaining the largest Euclidean distance by determining pairwise between a distal tip electrode and each of three coplanar electrodes to obtain three Euclidean distances and then selecting the largest of the three Euclidean distances.

In at least one embodiment, the geometric profile is obtained by forming a loop when plotting 3 electric field vector components of the electric field.

In at least one embodiment, the method comprises determining a change in the conduction wave direction by determining a change in angle of a direction axis of the electric field, where the angle is a projected angle of the electric field that is used to determine the EFSP.

In at least one embodiment, the conduction velocity is determined by taking a product of a direction vector of the electric field and a ratio of a time derivative of an average signal representing an electrode configuration over a sensed peak-to-peak bipole voltage, where the direction vector is a directional axis where a largest voltage of the electric field lies which is determined by rotation and projection of the electric field.

In at least one embodiment, the method comprises monitoring the conduction velocity over a desired span in terms of event counts or a specified time duration to track changes in the conduction velocity.

In at least one embodiment, the method further comprises using the EFSP as a voltage threshold parameter for a pacemaker/ICD unit.

In at least one embodiment, the method further comprises detecting changes in the electric field geometry to distinguish a change or shift in direction of a traveling wave due to different arrhythmogenic sources.

In at least one embodiment, the method comprises determining the electric field by: taking an amplitude difference between each unique pair of unipolar signals from each electrode; forming a spatial displacement matrix comprising the set of unique electrode pairs and their corresponding physical displacement coordinates; and determining a negative of a product between the set of derived bipoles and the inverse of the spatial displacement matrix.

In at least one embodiment, the method further comprises: detecting a first direction of an activated wave by aligning the activated wave with a first longest axis which is a first largest amplitude of a first electric field recorded during normal sinus rhythm; comparing the first direction with a second direction of a second longest axis which is a second largest amplitude of a second electric field recorded during pace-mapping; and providing a score of how similar the first direction is to the second direction.

In at least one embodiment, the method further comprises: recording the sensed signals as the EP data after signal capture occurs during a normal cardiac rhythm; storing a normal electric field that is derived from the recorded EP data during the normal cardiac rhythm; defining a normal template from an electric field geometry for the stored normal electric field that is associated with the normal cardiac rhythm; determining a matching score by taking a correlation of the normal template with electric field geometries from a later determined electric field for a given heart location; comparing the matching score to a matching score threshold to identify when the later determined electric fields are associated with an abnormal cardiac rhythm to identify changes in heart rhythm or morphologies that are different compared to the normal cardiac rhythm; and when the later determined electric field is abnormal, providing a pacing stimulus to induce normal cardiac rhythm.

In at least one embodiment, the method further comprises: recording the sensed signals as the EP data after signal capture occurs during an arrhythmia; storing an arrhythmia electric field that is derived from the recorded EP data during the arrhythmia; defining an abnormal template from a first electric field geometry for the stored arrhythmia electric field that is associated with the arrhythmia; determining a second electric field having a second electric field geometry resulting from pacing at a given heart location during a medical procedure with a roving ablation catheter; determining a matching score by taking a correlation of the first electric field geometry with the second electric field geometry; comparing the matching score to a matching score threshold to identify when the second electric field matches the arrhythmia electric field; and when the second electric field matches the arrhythmia electric field, indicating to a medical practitioner that a remedial action be taken comprising ablation or cryofreezing at the given heart location where the pacing by the roving ablation catheter caused the second electric field.

In at least one embodiment, the method further comprises using the electric field to maximize His detection by rotating the electric field to emphasize His-Bundle activity while suppressing cardiac muscle activation.

In at least one embodiment, the multi-electrode device is defined according to any of the embodiments described in accordance with the teaching herein.

In another broad aspect, in accordance with the teachings herein there is provided a method for providing cardiac pacing using a lower stimulus threshold using a pacemaker device and a multi-electrode device located at a heart location, the device having a 3D electrode configuration as defined in accordance with any of the teachings herein, and the method comprises: sensing voltages at the heart location using the electrodes; defining combinations of an anode and cathode for each combination of the electrodes, and for each combination of the electrodes and a body of the pacemaker device; determining sensed voltages for each of the anode and cathode combinations; determining a pacing stimulus for each of the anode and cathode combinations using the pacemaker; selecting the anode and cathode combination having the pacing stimulus with the lowest amplitude voltage; and using the selected anode and cathode combination to provide pacing stimuli to the heart.

In at least one embodiment, the method is repeated periodically to determine and use the anode and cathode combination having the pacing stimulus with the lowest amplitude voltage.

In at least one embodiment, the method is automatically repeated every day, every month, every quarter, every year or during follow up testing of the heart.

In another broad aspect, in accordance with the teachings herein there is provided a system for analyzing electrophysiological (EP) data from a 3D multi-electrode device having four electrodes positioned at a distal end of the device in a 3D tetrahedral configuration and the device being located at a heart, wherein the system comprises: a data store comprising program instructions stored thereon for executing methods; and at least one processor coupled to the data store, the at least one processor being configured to execute the program instructions to: sense unipolar voltages with the four electrodes individually to provide sensed signals; record the sensed signals as the EP data after signal capture occurs; determine an electric field from the EP data measured by the four electrodes; generate one or more features derived from the electric field; and analyze the features to determine when a heart rhythm of the heart has an irregular change, is erratic or is abnormal.

In at least one embodiment, the 3D multi-electrode device is defined in accordance with any of the teachings herein.

In at least one embodiment, the at least one processor is configured to determine at least one of an electric field span potential (EFSP), a geometric profile of the electric field, a travelling wave conduction direction and a conduction wave velocity as the one or more features derived from the electric field.

In at least one embodiment, the at least one processor is configured to determine the EFSP by determining a largest Euclidean distance formed with the electric field and scaling the Euclidean distance with an inter-electrode distance to express the EFSP as a voltage.

In at least one embodiment, the at least one processor is configured to determine the largest Euclidean distance pairwise between a distal tip electrode and each of three coplanar electrodes to obtain three Euclidean distances and then select the largest of the three Euclidean distances.

In at least one embodiment, the at least one processor is configured to obtain the geometric profile by forming a loop when plotting 3 electric field vector components of the electric field.

In at least one embodiment, the at least one processor is configured determine a change in a conduction wave direction by determining a change in angle of a direction axis of the electric field, where the angle is a projected angle of the electric field that is used to determine the EFSP.

In at least one embodiment, the at least one processor is configured to determine the conduction velocity by taking a product of a direction vector of the electric field and a ratio of a time derivative of an average signal representing an electrode configuration over a sensed peak-to-peak bipole voltage, where the direction vector is a directional axis where a largest voltage of the electric field lies which is determined by rotation and projection of the electric field.

In at least one embodiment, the at least one processor is configured to monitor the conduction velocity over a desired span in terms of event counts or a specified time duration to track changes in the conduction velocity.

In at least one embodiment, the at least one processor is configured to use the EFSP as a voltage threshold parameter fora pacemaker/ICD unit.

In at least one embodiment, the at least one processor is configured to detect changes in the electric field geometry to distinguish a change or shift in direction of a traveling wave due to different arrhythmogenic sources.

In at least one embodiment, the at least one processor is configured to determine the electric field by: taking an amplitude difference between each unique pair of unipolar signals from each electrode; forming a spatial displacement matrix comprising the set of unique electrode pairs and their corresponding physical displacement coordinates; and determining a negative of a product between the set of derived bipoles and the inverse of the spatial displacement matrix.

In at least one embodiment, the at least one processor is configured to detect a first direction of an activated wave by aligning the activated wave with a first longest axis which is a first largest amplitude of a first electric field recorded during normal sinus rhythm; comparing the first direction with a second direction of a second longest axis which is a second largest amplitude of a second electric field recorded during pace-mapping; and providing a score of how similar the first direction is to the second direction.

In at least one embodiment, the at least one processor is configured to record the sensed signals as the EP data after signal capture occurs during a normal cardiac rhythm; store a normal electric field that is derived from the recorded EP data during the normal cardiac rhythm; define a normal template from an electric field geometry for the stored normal electric field that is associated with the normal cardiac rhythm; determine a matching score by taking a correlation of the normal template with electric field geometries from a later determined electric field for a given heart location; compare the matching score to a matching score threshold to identify when the later determined electric fields are associated with an abnormal cardiac rhythm to identify changes in heart rhythm or morphologies that are different compared to the normal cardiac rhythm; and when the later determined electric field is abnormal, provide a pacing stimulus to induce normal cardiac rhythm.

In at least one embodiment, the at least one processor is configured to record the sensed signals as the EP data after signal capture occurs during an arrhythmia; store an arrhythmia electric field that is derived from the recorded EP data during the arrhythmia; define an abnormal template from a first electric field geometry for the stored arrhythmia electric field that is associated with the arrhythmia; determine a second electric field having a second electric field geometry resulting from pacing at a given heart location during a medical procedure with a roving ablation catheter; determine a matching score by taking a correlation of the first electric field geometry with the second electric field geometry; compare the matching score to a matching score threshold to identify when the second electric field matches the arrhythmia electric field; and when the second electric field matches the arrhythmia electric field, indicate to a medical practitioner that a remedial action be taken comprising ablation or cryofreezing at the given heart location where the pacing by the roving ablation catheter caused the second electric field.

In at least one embodiment, the at least one processor is configured to use the electric field to maximize His detection by rotating the electric field to emphasize His-Bundle activity while suppressing cardiac muscle activation.

In another broad aspect, in accordance with the teachings herein there is provided a system for providing cardiac pacing using a lower stimulus threshold using a pacemaker device and a multi-electrode device located at a heart location, the device having a 3D tetrahedral configuration, wherein the system comprises: a data store comprising program instructions stored thereon for executing methods; and at least one processor coupled to the data store, the at least one processor being configured to execute the program instructions to: sense voltages at the heart location using the electrodes; define combinations of an anode and cathode for each combination of the electrodes, and for each combination of the electrodes and a body of the pacemaker device; determine sensed voltages for each of the anode and cathode combinations; determine a pacing stimulus for each of the anode and cathode combinations using the pacemaker; select the anode and cathode combination having the pacing stimulus with the lowest amplitude voltage; and use the selected anode and cathode combination to provide pacing stimuli to the heart.

In at least one embodiment, the at least one processor is configured to periodically determine and use the anode and cathode combination having the lowest amplitude voltage for providing pacing stimuli to the heart.

In at least one embodiment, the at least one processor is configured to repeated periodically determine and use the anode and cathode combination having the pacing stimulus with the lowest amplitude automatically every day, every month, every quarter, every year or during follow up testing of the heart.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows an illustration of an example embodiment of a 3D multi-electrode device showing how the device is structured from a distal to proximal end.

FIG. 1B shows an illustration of an outer view of another example embodiment of a 3D multi-electrode device having a wireless configuration and being implantable.

FIG. 1C shows an illustration of an interior view of another example embodiment of a 3D multi-electrode device having a wireless configuration and including a miniaturized pacemaker/ICD unit.

FIG. 2A shows a top view of an example embodiment of a 3D electrode configuration at a tip of the device of FIGS. 1A-1C, the view showing the electrode orientation from the tip perspective.

FIG. 2B shows a second view of the electrode configuration of FIG. 2A, the view showing a tetrahedral configuration formed by the electrodes.

FIG. 2C shows a third view of the electrode configuration of FIG. 2A, the view showing how the wiring is attached to each electrode from the interior.

FIG. 3A shows an example embodiment of a prototype of a device according to the embodiment of FIG. 1A, showing a connector pin and a tip with a 3D electrode configuration in accordance with the teachings herein.

FIG. 3B shows a close-up view of the 3D electrode configuration at a distal end of the prototype device of FIG. 3A.

FIG. 4A shows an illustration of an example embodiment of a pacemaker device with a lead having a 3D electrode configuration in accordance with the teachings herein where the lead tip is positioned in the right ventricle (RV) apex for sensing cardiac signals.

FIG. 4B shows an illustration of an example embodiment of a pacemaker with a lead having a 3D electrode configuration in accordance with the teachings herein where the lead tip is positioned on a surface of the right atrium (RA) for sensing cardiac signals.

FIG. 5 shows an illustration of an example embodiment of a wireless device having a 3D electrode configuration in accordance with the teachings herein where the wireless device is implanted into a surface of the heart.

FIG. 6 is a block diagram of an example embodiment of a mapping system for use with a 3D multi-electrode device in accordance with the teachings herein.

FIG. 7 shows a flow chart of an example embodiment of a method of acquiring and analyzing electrophysiology data using a 3D multi-electrode device in accordance with the teachings herein.

FIG. 8 shows a flow chart of another example embodiment of a method of acquiring and analyzing electrophysiology data using a 3D multi-electrode device in accordance with the teachings herein.

FIG. 9A shows a flow chart of an example embodiment of a method of using an electric field measured by a multi-electrode device having a 3D electrode configuration in accordance with the teachings herein.

FIG. 9B shows a flow chart of an example embodiment of a method of providing cardiac pacing using a multi-electrode device having a 3D electrode configuration in accordance with the teachings herein.

FIG. 10A shows graphs of examples of traditional bipolar combination signals (Bi-1 to Bi-6) for a heartbeat.

FIG. 10B shows a graph of an example Electric-field span potential (EFSP) for the same heartbeat graphed in FIG. 10A.

FIG. 11 shows an example boxplot comparing voltages from traditional bipolar combinations and the EFSP while sensing in the Right Ventricle (RV) apex during sinus rhythm.

FIG. 12 shows an example boxplot comparing voltages between traditional bipolar combinations and the EFSP while sensing in the Right Atrium (RA) during sinus rhythm.

FIG. 13A shows an example loop graph determined from sensing cardiac signals from the RV apex while pacing from the Left Atrial Appendage (LAA).

FIG. 13B shows an example loop graph determined from sensing cardiac signals from the RV apex while pacing from the Left Ventricle (LV) septum.

FIG. 14A shows an illustration of an example simulation of a wave activation travelling to the right.

FIG. 14B shows an illustration of an example Electric-field produced by an advancing wave relative to the surface of the myocardium in an in-vivo environment.

FIG. 15A shows an illustration of an example electric field loop produced by a wave activation.

FIG. 15B shows an illustration of an example of multicomponent bipolar electrograms.

FIG. 16A shows an illustration of a multi-axes lead deployed in the apex of a right ventricle form where SR, LV pacing, and VF was recorded.

FIG. 16B shows an illustration of a left bundle in a fresh sheep LV septal slab.

FIG. 16C shows an illustration of conduction signal capture proved by the presence of left bundle potential preceding the local EGM.

FIG. 17 shows an illustration of the six sensed bipolar electrograms used to form a 3D Electric-field loop, the resultant Electric-field loop, and the resultant EFSP EGM.

FIG. 18 shows an illustration of the Electric-field vector components and the 3D Electric-field that can be formed therefrom at three different angles.

FIG. 19 shows an illustration of an experimental setup and electrode configuration used to observe the difference in sensed voltages between the bipolar combinations and the EFSP, and the corresponding conditions during sinus rhythm, pacing from right ventricle, pacing from left ventricle, and ventricular fibrillation.

FIG. 20 shows example charts of voltages of bipolar configuration and EFSP during different rhythms.

FIG. 21 shows example loop graphs of the electric field for three beats during sinus rhythm, pacing in the RV, and pacing in the LV.

FIG. 22 shows example charts of voltage thresholds in two animals for multi-axes pacing at RV apex.

FIG. 23 shows an example graph of left bundle pacing threshold measured from different bipoles of the multi-axes lead in seven animals.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

The example embodiments of the devices, systems, or methods described in accordance with the teachings herein may be implemented as a combination of hardware and software. For example, at least some embodiments or a portion of the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise input devices including at least one of a touch screen, a keyboard, a mouse, buttons, keys, sliders, and the like, as well as one or more of a display, a printer, and the like depending on the implementation of the hardware.

It should also be noted that there may be some elements that are used to implement at least part of the embodiments described herein that may be implemented via software that is written in a high-level procedural language such as object oriented programming. The program code may be written in MATLAB, C, C⁺⁺, or any other suitable programming language and may comprise modules or classes, as is known to those skilled in object oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having at least one processor, an operating system, and the associated hardware and software that is necessary to implement the functionality of at least one of the embodiments described herein. The software program code, when read by the device, configures the device to operate in a new, specific, and predefined manner (e.g., as a specific purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

In accordance with the teachings herein, there are provided various embodiments for devices and methods for an intelligent vector (“iV”) technology which may be used with pacemakers and ICDs, for example, where the iV technology incorporates a wired lead or is wireless and has a novel electrode configuration.

In at least one embodiment, the electrode configuration allows for detection of the largest bipoles by electronic repositioning of a single lead tip, instead of relying on mechanical manipulation of bipoles to relocate the lead both in the atrium and the ventricle; this is similar for the wireless electrode embodiments described herein. A technical problem addressed by the teachings herein is that when a sensed voltage drops, which frequently requires manual lead adjustments, the novel electrode configuration can be electronically repositioned to increase the sensed voltage which results in more reliable voltage sensing in both the atrium and the ventricle by maximizing voltage sensing in a vectorial plane, which is based on a physical layout of the electrodes that form the base of a tetrahedral structure. The electrode pairing is electronically repositioned as the electric field that is derived from the voltages sensed by the electrodes is rotated and projected to find the largest voltage. As the voltage value is being updated at a rate specified by a programmed processing unit, the rotation and projection of the electric field will occur and result in the electronic repositioning of the electrodes.

In at least one embodiment, the electrode configuration allows the user to differentiate signals emerging from one of the atriums, the bundle of His, and one of the ventricles, as that is a major limitation for conventional His pacing leads. Having characterized the signal sources, the user is able to distinguish between signals for device programming. This is advantageous since a technical problem with conventional leads is that they cannot be used to differentiate far-field signals from local signals and as such these signals may be confused with one another. In contrast, in accordance with at least one of the embodiments described herein, the novel electrode configuration can be used for signal source categorization, as will be described in further detail below.

In at least one embodiment, when there are arrhythmias, such as ventricular fibrillation and/or atrial fibrillation, and the signal amplitude that is measured by a conventional lead drops, the novel electrode configuration described herein can be used to record larger signal readings and a more definite result for the maximum iV electrode technology voltage (i.e. the largest voltage that represents the electric field). This is due to the the tetrahedral electrode configuration, which provides better sensing of larger voltages by using additional electrodes in a specified region and determining a 3-D component aspect to the electric field. This is advantageous since during arrhythmias, the sensed signal amplitude can drop, making detection between the noise floor and arrhythmia electrograms difficult, and the novel electrode configuration can be used to provide a more reliable and larger signal reading when arrhythmias are present.

Changes in rhythm from a regular rhythm to ventricular tachycardia (VT) or a regular supra ventricular rhythm and detection, differentiation and treatment of the VT and detection of bundle branch allow for commencement of cardiac resynchronization as needed.

The pacemaker and/or ICD device, or other cardiac electronic devices, along with the novel electrode configurations described in accordance with the teachings herein can function as a mapping system in which electrogram matching, described in further detail below, is used to provide a better mapping of the arrhythmia origin by detecting the direction of the wavefront, thereby allowing for pace-mapping of arrhythmogenic focus.

Another technical problem is that when the cardiac rhythm changes, it is difficult to differentiate from a normal sinus rhythm, and there is a loss of signal capture when using a conventional LV lead. However, in accordance with at least one embodiment described herein, the novel electrode configuration can be used to detect changes in heart rhythm by measuring Conduction Velocity (CV) on a beat-to-beat basis to allow for treatment planning of VT or cardiac resynchronization. Measurement of CV is useful because a change in CV reflects a change in heart rhythm. Conventional leads are equipped with electrodes positioned collinearly along the lead's shaft and thus cannot determine CV. Conventional leads do not have a spatial configuration and, in principle, they cannot provide a high enough signal quality due to the material used to fabricate these leads. This problem can be overcome with the electrodes described herein which can be arranged in such a way that they form the vertices of a volume (e.g., a tetrahedron) which allows for identifying changes in CV. As a wave direction can be determined, the CV can be derived by taking the product of a direction vector and the ratio of a time derivative over a sensed bipole voltage. The direction vector is the result of the directional axis where the largest voltage lies (this is the largest voltage obtained from the rotation and projection of the electric field). This axis can be represented as a unit vector in the coordinate system defined by the electrodes. The ratio of the time derivative over the sensed bipole voltage can be obtained by determining a change in the average signal that represents the electrode configuration, dividing it by the elapsed time and taking the peak-to-peak voltage. By monitoring the CV over a desired span either in terms of event counts or a specified time duration, the changes in CV can be tracked.

Another technical problem is that when patients with ICDs with existing conventional leads have VT, while this can be recorded, by the time that the patient is treated by ablation of the focus, the focus may not fire during the ablation procedure. If the surgical operator can rove and create an activation from a similar site, then an electrogram for the activated wave can be recorded and compared to a previously recorded electrogram on the lead, which allows the focus to be recreated when there is perfect match between these two electrograms. However, conventional leads are incapable of doing this. Advantageously, at least one of the embodiments described in accordance with the teachings herein provides a technical solution to this problem because the novel electrode configuration can be used to detect the direction of an activated wave by aligning with the longest axis (i.e. largest amplitude) of an electric field or loop to be used for pace-mapping of the arrhythmogenic focus so that ablation can be accurately delivered at the correct location at the time of the procedure. This may be done, for example, by deriving a set of unique bipoles by taking the amplitude difference between each unique pair of unipolar signals from each electrode, under the assumption that the overall set of electrodes are not co-linear. A spatial displacement matrix may then be formed containing the set of unique electrode pairs and their corresponding physical displacement coordinates. These coordinates are formed with respect to the 3D geometry formed by the electrode's x, y, z coordinate system for the tetrahedral electrode configuration. The electric field is then determined by taking the negative of the product between the set of derived bipoles and the inverse of the spatial displacement matrix.

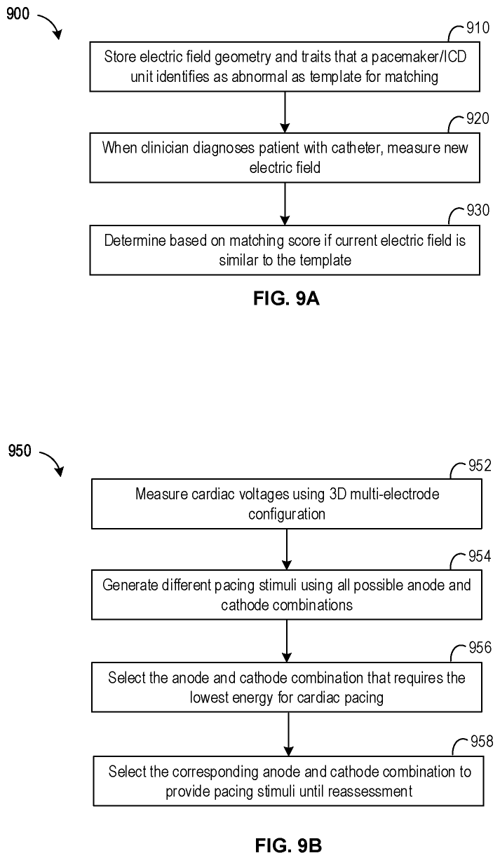

Furthermore, the application of different voltages in a single lead, by applying different potentials across independent electrodes, can be utilized in the stimulation of the His bundle, leading to efficient Purkinje capture with minimal current drain. Such stimulation of the His bundle is an aspect of pacing similar to sensing done in multiple axes, which may allow for identification of better pacing efficiency. Better pacing efficiency means that a combination of electrodes is determined that requires the least amount of voltage to provide a pacing stimulus to the heart. An example embodiment of a method for performing more efficient pacing is described in further detail with respect to FIG. 9B.

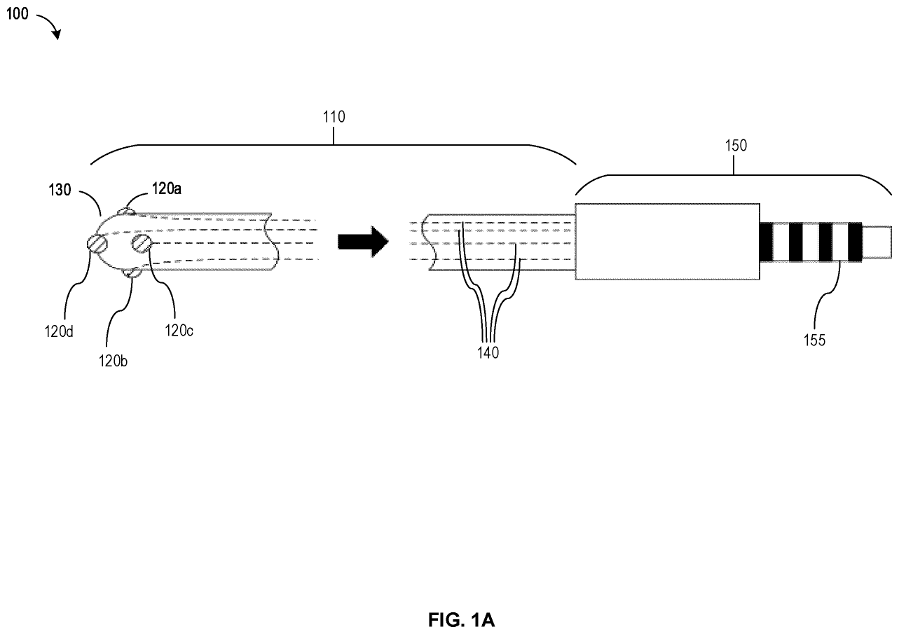

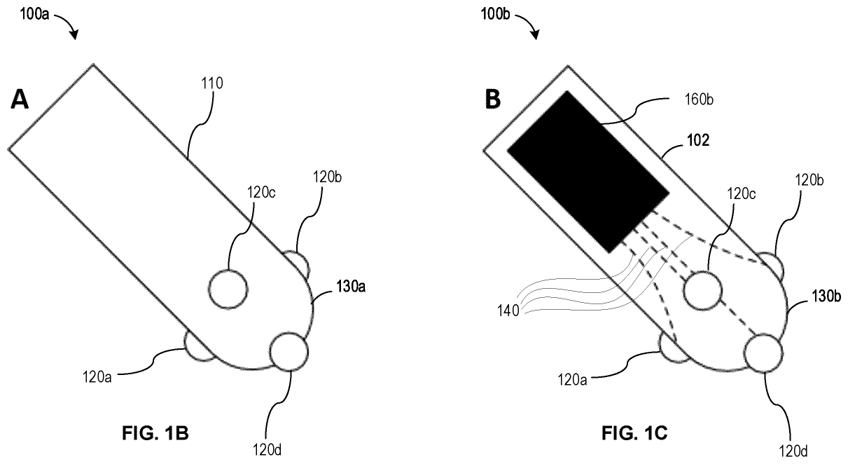

Reference is first made to FIG. 1A, showing an illustration of an example embodiment of a 3D multi-electrode device 100, showing the structure of the device from distal to proximal end. It should be noted that while the device 100 is described for use with a pacemaker and/or an ICD herein, it may be used in other applications as well. The device 100 may be alternatively referred to as a multi-axes lead as in, for example, the discussion of Study 2 below.

The device 100 can be used as part of an iV technology for pacemaker/ICDs due to the ability for the 3D electrode configuration of the device 100 to sense certain voltages and be used to measure certain physiological characteristics including an electric field that results from the voltages sensed by the electrodes.

The device 100 comprises a lead 110 and a tip 130 having four electrodes 120a-120d that are oriented in a tetrahedral configuration. Individual wires 140 extend from the electrodes 120a-120d to an output. The output may be an IS4 connector 150 having a connection pin 155. The device 100 provides a better means of detecting the maximum bipolar voltage for monitoring the condition of the heart regardless of any change in position of the lead 110 or tip 130.

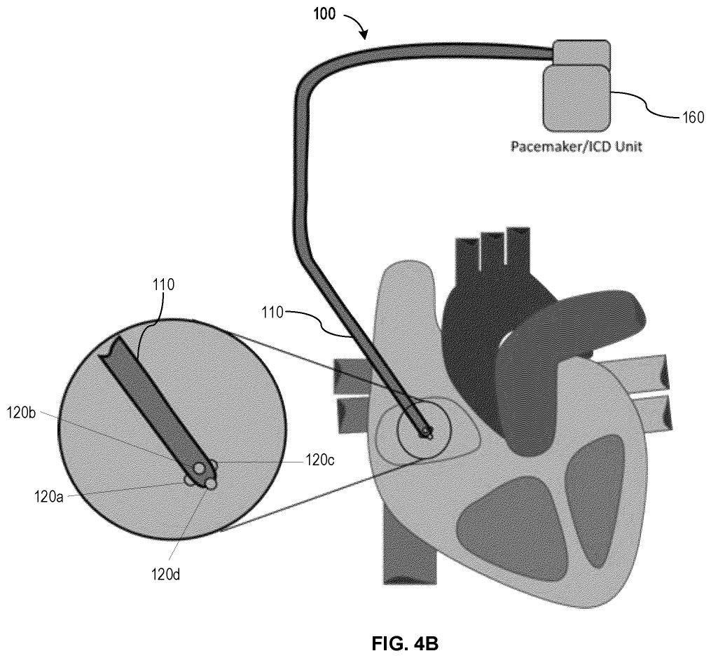

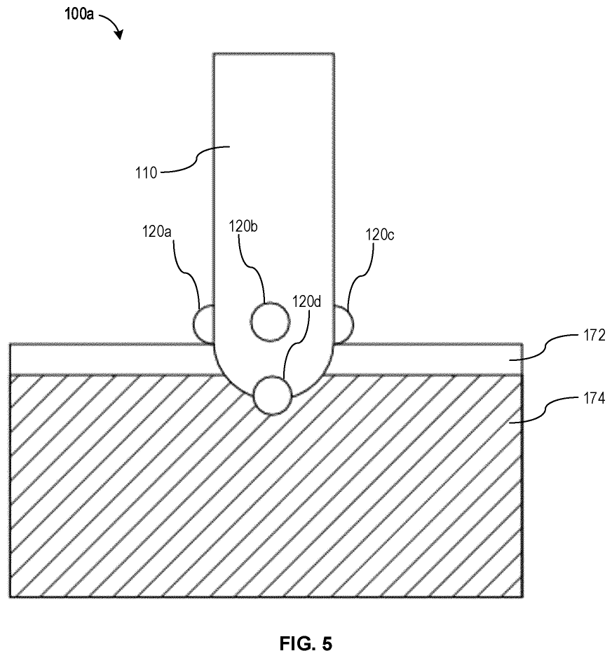

In general, positioning of a lead with conventional electrodes is important for allowing a pacemaker/ICD unit 160 (shown in FIGS. 4A-4C and 5), that is connected to the conventional lead, to receive sensed voltages with good Signal to Noise Ratio (SNR) in order to make appropriate decisions when the patient's heart rhythm becomes abnormal. Advantageously, any error that the pacemaker/ICD unit 160 makes may be reduced by ensuring that a satisfactory level of voltage (i.e. satisfactory SNR) is detected from any pair of the electrodes 120a-d by securing the device tip 130 properly in the ventricle.

For example, the physical positioning of the electrodes of a conventional lead may be shifted due to heart motion. This difference in positioning will induce an offset to the voltage reading and will cause misdetection and misdiagnosis for any device that uses the signals provided by the conventional lead for detecting and/or diagnosing certain heart conditions. Advantageously, the voltage signals sensed by the device 100 can be used to derive an electric field due to the tetrahedral configuration of the electrodes 120a-120d and obtain a peak-to-peak voltage based on the derived electric field. As the electric field is more stable in comparison to the direction-dependent bipolar voltages, it reduces the concern for minor shifts of the positioning of the tip 130 as the voltage reading derived from the electric field is still acceptable. Since the configuration of the electrodes 120a-120d reduces voltage changes due to shifts in positioning of the tip 130, the device 100 does not require repositioning by a surgeon, which implies fewer surgical operations for the patient.

The electrodes 120a-120d can be handmade by utilizing thermal techniques local to certain positions of the device 100. For example, using silver wires, heat can be applied to the tip of the wires 140 and removed once a sphere (not shown) is produced having the desired size. The spheres form the actual electrodes 120a-120d. Aside from the electrodes 120a-120d and the IS4 connector 150, all other parts of the device 100 can be purchased from an electrical hardware store. The IS4 connector 150 can be ordered from vendors.

Referring now to FIG. 1B, shown therein is an illustration of an outer view of an example embodiment of a device 100a with a wireless configuration. The electrodes 120a-120d may be made in the same manner as was described for the device 100 and disposed in the same geometrical configuration at tip 130a. The device 100a houses a communication unit (not shown) such as, but not limited to, a Bluetooth unit, for example, for transmitting the sensed cardiac signals to another device that preprocesses and/or analyzes the cardiac signals.

Referring now to FIG. 1C, shown therein is an illustration of an interior view of an example of an alternative embodiment of a device 100b with a wireless configuration that includes an implantable pacemaker. In this embodiment, the device 110b may be referred to as a “capsule unit”, as it houses some or all of the electronics of a miniaturized pacemaker/ICD unit 160b (for instance, examples of such miniaturized devices include the Medtronic Micra and the Abbott Nanostim). Accordingly, the device 110b includes a shell or housing 102, a pacemaker/ICD unit 160, and electrodes 120a-120d that are coupled to the pacemaker/ICD unit 160 via wires 140. The electrodes 120a-120d, wires 140, and tip 130b can be made as was described for lead device 100. The miniaturized pacemaker/ICD unit 160 may be housed in a distal portion (e.g., one half) of the capsule unit 100b. The device 100b preserves the tetrahedral configuration of the electrodes 120a-120d (e.g., at the tip 130b, i.e. proximal first half).

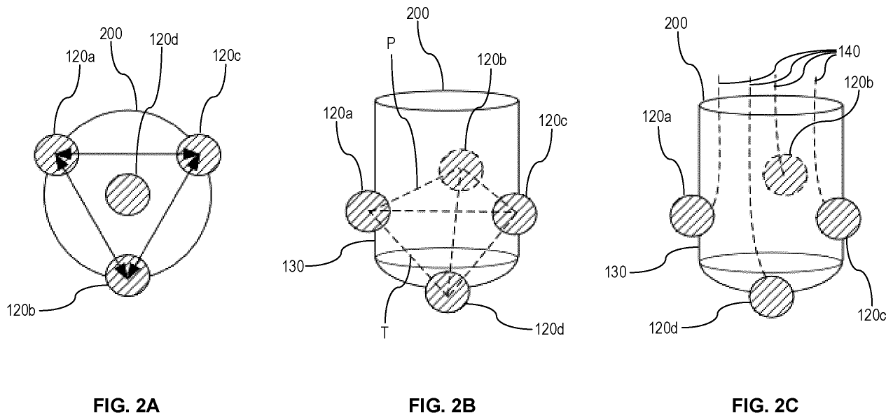

Referring now to FIG. 2A, shown therein is a first view of an example of an electrode configuration 200 at a device tip, the view showing the electrode orientation from the tip perspective without any wires connected to the electrodes 120a-120d. The first three electrodes 120a-120c (of the four electrodes) are arranged in a triangular configuration, which may be an equilateral triangle, in a plane that is orthogonal to a longitudinal axis of the device. The fourth electrode 120d is positioned vertically spaced away from the plane and aligned with the centre of the triangle and the longitudinal axis of the device, as viewed from above the tip (i.e. from the bottom of FIGS. 2B and 2C).

Referring now to FIG. 2B, shown therein is a second view of an example of an electrode configuration 200 at a device tip, the view showing a tetrahedral configuration T formed by the electrodes 120a-120d. The first three electrodes 120a-120c (of the four electrodes) are in one plane P, which may be coplanar with a circular cross-section of a lead 110 to which the device may be connected. The fourth electrode 120d is positioned at the tip, outside the plane P of the first three of the four electrodes. Together, the four electrodes 120a-120d may make the vertices of a tetrahedron T, which may be a regular tetrahedron.

When using only 3 electrodes arranged in a plane, only a 2-D projection of the electric field can be derived. However, the 3D electrode configuration 200 enables derivation of an electric field in 3-D due to the voltages measured by each of the electrodes 120a-120d as the fourth electrode 120d provides the vertical aspect of the electric field. In the instance of using only the three electrodes 120a-120c in an equally spaced triangular planar configuration, it would only detect propagating waves travelling along the surface of the heart. However, the tetrahedral configuration T of electrodes 120a-120d provides a better estimation of the travelling wave for cases where the travelling wave comes from underneath the electrodes 120a-120d and at different angles with respect to the plane formed by the electrodes 120a-120c.

Referring now to FIG. 2C, shown therein is a third view of an example of an electrode configuration 200 at a device tip, the view showing how the wiring is attached to each electrode 120a to 120d from the interior. Each of the four individual wires 140 are attached to only one of the corresponding four electrodes 120a to 120d and run through the inside of the lead device. Alternatively (not shown), one or more of the individual wires 140 can run along or near the surface of the lead device 110.

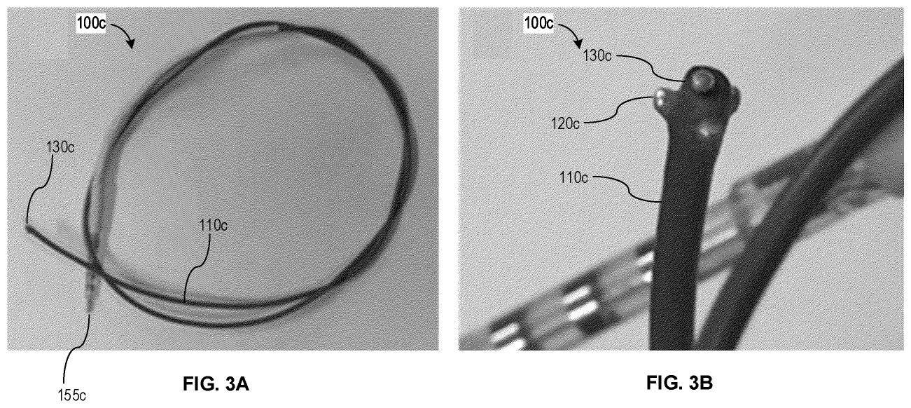

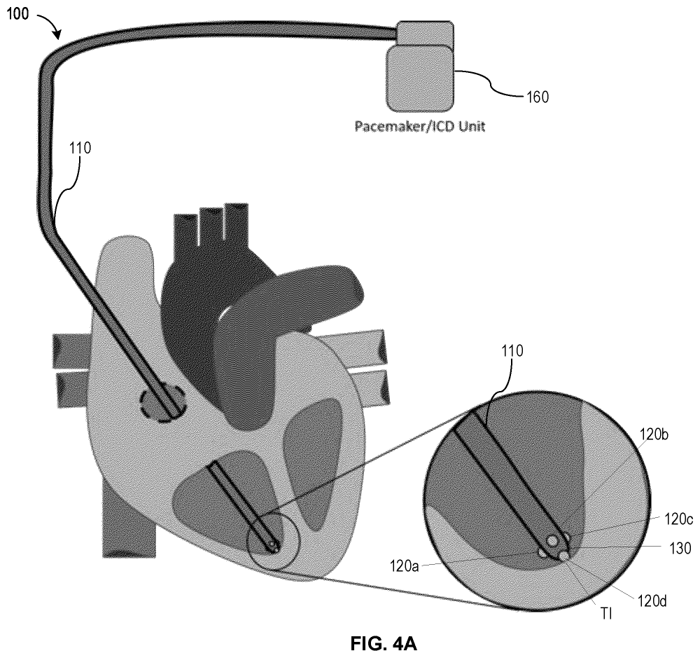

Referring now to FIGS. 3A and 3B, shown therein is an example embodiment of a prototype of a device 100c, showing the tip 130c, the lead 110c, and the end connector pin 155c. FIG. 3B shows a close-up view of the electrode configuration at a distal end of the device 100c. Only one of the four electrodes 120c is labeled so as not to clutter the image.

While making reference to device 100 for illustrative purposes, it should be understood that the following description is applicable to the other embodiments herein. The device 100 has a set of electrodes 120a-120d that are configured and positioned at a tip 130 of the device 100 for better reliability in detecting bipolar voltages. In general, the device 100 can have four identically sized, spherical electrodes 120a-120d made from the same material embedded in an equally spaced tetrahedral configuration at the distal end; four individual wires 140 extending from corresponding electrodes 120a-120d to an output; and a single connector pin at the proximal end that is connected to the output and can be used to transmit the electrophysiological signals sensed from the electrodes 120a-120d to a pacemaker/ICD unit 160 or another device, to perform signal analysis for monitoring and/or treatment. The four electrodes 120a-120d are disposed at an end of the device and are arranged in a tetrahedral configuration. The electrode 120d that is at the top vertex of the tetrahedron aligns with the tip of the end of the device 100 such that this electrode 120d (i.e., the distal tip electrode) is disposed along a longitudinal central axis of the end portion of the device 100. Aligning the distal tip electrode 120d along the longitudinal central axis can ensure that the interelectrode distance is equal between the tip electrode 120d and the remaining three electrodes 120a-120c. Such alignment and distance control may be advantageous for the post processing of the resulting electrogram signals. For example, by having an equal distance between all electrodes, the derived bipoles will not be affected differently by distance since they are equidistant with respect to one another. If alignment and distances varied, then the electrode that provides the best sensing will be tracked.

In at least one embodiment, the devices 100-110c can have three of the four electrodes positioned to form the equilateral triangular base (which may alternatively be referred to as a “coplanar configuration”) of the tetrahedral electrode configuration. In a given embodiment, the three electrodes 120a-120c can all be equally spaced in relation to the distal tip electrode 120d and in relation to one another. In a given embodiment, the size of the electrodes 120a-120d is the same. In alternative embodiments, electrode spacing and sizing may be altered depending on the size of the device tip 130. In any given embodiment, the size of the electrodes 120a-120d is dependent on the trade-off between spatial resolution (e.g., use of smaller electrodes results in better resolution) and signal strength (e.g., use of larger electrodes results in more surface area).

In at least one embodiment, the devices 100, 100c can have an extension of a wire from each individual electrode to the end of the lead 110, 110c where the wires are coupled to an output which in turn may be coupled to a connector.

In at least one embodiment, the devices 100, 100c can have a shaft made of soft material encompassing the lead 110, 110c to allow for maneuverability of the devices 100, 100c when they are placed with the heart and to also accommodate for motion of the heart after the tips 130, 130c of the devices 100, 100c have been placed at a certain location in the heart.

In at least one embodiment, the lead devices 100, 100c have wires with proximal ends that can be connected to industry standard pins, which may, for example, be compatible with existing pacemaker/ICD units.

In at least one embodiment, the electrodes are spheres with diameter of 1 mm to 2 mm. Although other three-dimensional shapes are possible, spherical electrodes give better surface area for contact as the tissue surface is not uniform. Diameters less than 1 mm and greater than 2 mm may also be used, for example, for electrodes made with material that is more or less conductive (the conductivity of the material can have an effect on signal quality) or for hearts of different sizes (e.g., bigger hearts may require larger sized electrodes).

In at least one embodiment, the inter-electrode distance is the same and may range from about 2 mm to 5 mm. Inter-electrode distances less than 2 mm and greater than 5 mm may also be used, for example, for electrodes made with material that is more or less conductive or for use with hearts that are bigger or smaller as explained previously.

Referring now to FIG. 4A, shown therein is an illustration of an example embodiment of the device 100 with the tip 130 positioned in the right ventricle (RV) apex of a heart for sensing cardiac signals therefrom. With an industry standard connector pin, the device 100 integrates with, for example, a conventional pacemaker/ICD unit 160. The electrode 120d at the tip 130 is embedded into tissue TI while the remaining electrodes 120a-120c (which may be considered the “planar section” of the tetrahedral configuration) is adjacent to the tissue surface, an example of which is shown in FIG. 5. Alternatively, in at least one embodiment, a wireless device may be used, such as the device 100a as shown in FIG. 1B. In another alternative, the lead device 100b with a capsule unit including a miniaturized pacemaker/ICD unit 160b may be used in which case the larger pacemaker/ICD unit 160 is not used and the electrode 120d does not penetrate through the cardiac tissue but rather is adjacent to the surface of tissue.

Referring now to FIG. 4B, shown therein is an illustration of an example embodiment of a lead device 100 positioned on the right atrium (RA) for sensing. With an industry standard connector pin, the lead device 100 integrates, for example, with conventional pacemaker/ICD unit 160.

Alternatively, in at least one embodiment, a wireless device may be used such as the device 100a as shown in FIG. 1B. In another alternative, a wireless device 100b with a capsule unit including a miniaturized pacemaker/ICD unit 160b may be used in which case the larger pacemaker/ICD unit 160 is not used. It should be noted that such wireless embodiments may be useful for performance testing of the tetrahedral electrode configuration due to limited access in a Langendorff experiment setup.

Referring now to FIG. 5, shown therein is an illustration of an example embodiment of a wireless configuration of the device 100a when implanted. The wireless lead device 100a is placed such that at least one of the electrodes 120a-120c makes contact with the endocardium 172 and at least the electrode 120d (and possibly one or more of the electrodes 120a-120c) makes contact with the myocardium 174.

In at least one embodiment, the wireless device 100b can include an internal miniature pacemaker/ICD unit 160b, and the lead device 100b is situated at the area of interest in the heart (e.g., in a particular ventricle or atria) where pacing signals are to be provided.

In at least one embodiment, the wireless device 100a may be implanted as shown in FIG. 5 and has a wireless transmitter that can transmit the sensed signals to a separate device that may also be implanted, such as a subcutaneous ICD. The separate device has a wireless receiver for receiving the transmitted signals.

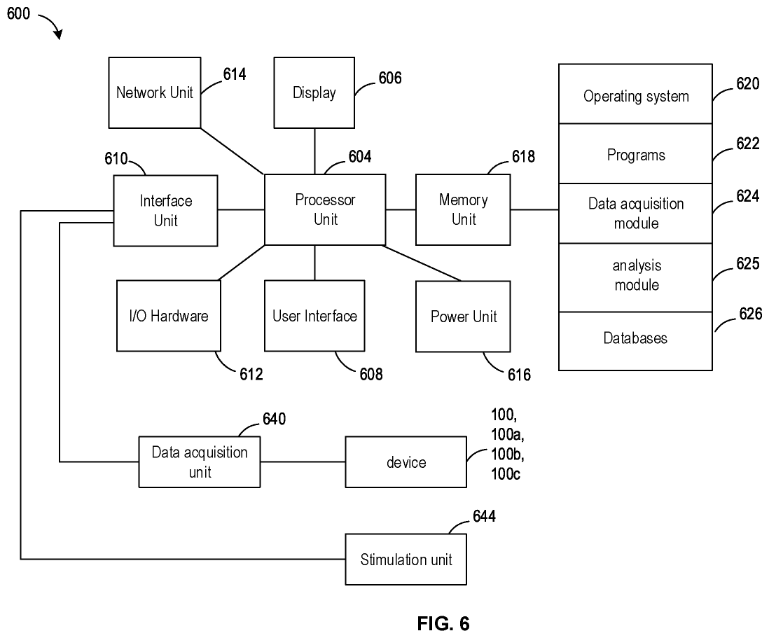

Referring now to FIG. 6, shown therein is a block diagram of an example embodiment of a mapping system 600 for use with the devices 100, 100a, 100b, or 100c. The system 600 includes a data acquisition unit 640 and a stimulation unit 644. The system 600 further includes a processor unit 604, a display 606, a user interface 608, an interface unit 610, input/output (I/O) hardware 612, a network unit 614, a power unit 616, and a memory unit (also referred to as “data store”) 618.

In general, a user may interact with the system 600 to obtain sensed signals from the device 100, 100a, 100b or 100c and process these sensed signals. Accordingly, the system 600 may be an electronic device or system that is used by the user. Alternatively, the system 600 may be a pacemaker or ICD device that may or may not be miniaturized. The system 600 is provided as an example, and there can be other embodiments of the system 600 with different components or a different configuration of the components described herein.

The processor unit 604 controls the operation of the system 600 and can be any suitable processor, controller, or digital signal processor that can provide sufficient processing power depending on the configuration, purposes, and requirements of the system 600 as is known by those skilled in the art. The processor unit 604 may include a standard processor, such as the Intel Xeon processor, for example. Alternatively, there may be a plurality of processors that are used by the processor unit 604, and these processors may function in parallel. Therefore, the processor unit 604 is considered as having at least one processor.

The user interface 608 may be used to generate a set of windows or graphical user interface (GUI) screens that can be used to display certain information to a user and receive input parameters from the user. Alternatively, the user interface 608 may include input devices that a user can use to provide data inputs or control inputs to the system 600. These input devices include a keyboard, a mouse, a touchscreen, and the like. The user interface 608 can also include devices to provide an output to the user, such as the display 606 or a printer. The display 606 may be, but is not limited to, a computer monitor, an LCD display, or a touchscreen monitor. When the system 600 is a pacemaker or ICD unit, the user interface 608 and the display 606 are not used and the user can interact with the system 600 via other means such as the network unit 610.

The interface unit 610 can be any interface that allows the system 600 to receive data from or send control signals to other devices such as the device 100, 100a, 100b or 100c and the stimulation unit 644. In some cases, the interface unit 610 can include at least one of a serial port, a parallel port, a USB port that provides USB connectivity, a wireless unit (as described below) when interacting with interacting with wireless device 100a or 100b, or another suitable port or connections for sending and receiving signals.

The network unit 614 may be a standard network adapter such as an Ethernet or 802.11x adapter or another type of adapter. Accordingly, the interface unit 610 can also include at least one of an Internet connection, a Local Area Network (LAN) connection, an Ethernet connection, a FireWire connection, a modem connection, or a digital subscriber line connection. Alternatively, or in addition, the network unit 614 may be a wireless unit. As a wireless unit, the network unit 614 can be a radio that communicates utilizing CDMA, GSM, GPRS, or Bluetooth protocol according to standards such as those in the IEEE 802.11 family (e.g., 802.11ac). The network unit 614 can be used by the operator unit 602 to communicate with other devices or computers.

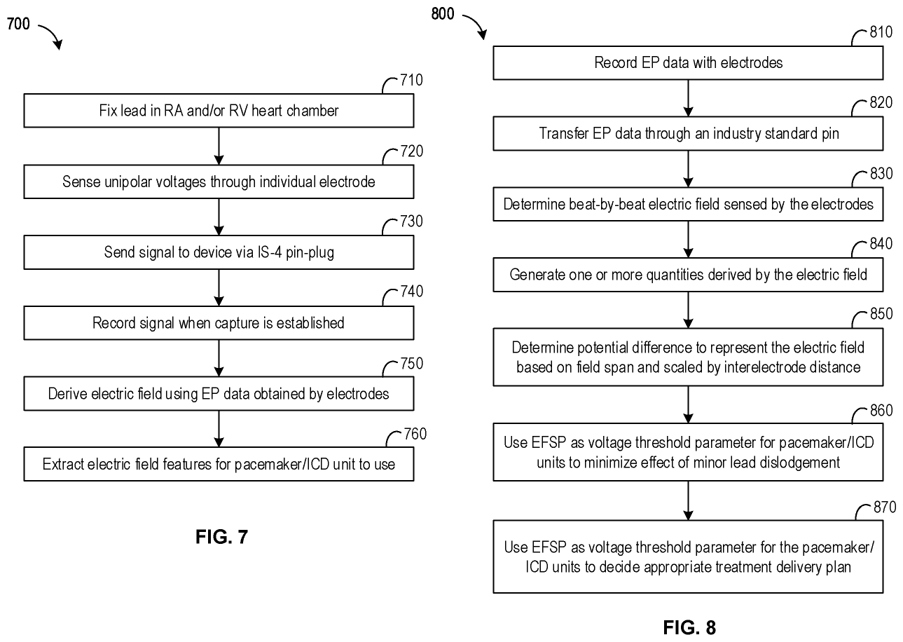

The memory unit 618 may store the program instructions for an operating system 620, program code 622, a data acquisition module 624, and one or more databases 626. The programs 622 comprise program code that, when executed, configures the processor unit 604 to operate in a particular manner to implement various functions and tools for the pacemaker lead device 100. The data acquisition module 624 comprises program code that may be used to obtain sensed signals and store these sensed signals in a database 626. For example, the data acquisition module 624 may comprise instructions for performing certain functions that are part of method 700 (see FIG. 7) or method 800 (see FIG. 8). The analysis module 625 comprises program code that may be used to analyze the sensed signals and provide cardiac data to a user of the system 600. For example, the analysis module 625 may comprise program code for performing some of the functions described in one or more of method 700, method 800, method 900 and method 950 (see FIGS. 7, 8, 9A and 9B).

The data acquisition unit 644 may be used to preprocess cardiac data that is sensed using the device 100, 100a, 100b or 100c from a patient or subject, which may be done in response to certain stimuli. Accordingly, the data acquisition module 624 may also be used to control the timing for stimulus generation and data acquisition. The data acquisition unit 644 comprises hardware circuitry that is used to record data sensed by the lead devices 100, 100a, or 100b from a patient or subject. For example, the data acquisition unit 644 may contain at least one amplifier, at least one filter, and an Analog Digital Converter (which may have multiple channels) for amplifying, filtering, and digitizing the sensed cardiac signals. Conventional amplification and filtering may be used. In cases where the system 600 is a conventional sized or miniaturized pacemaker or ICD unit, the data acquisition unit 644 is included in the pacemaker or ICD unit.

The stimulation unit 644 is used to provide stimulus signals to the heart of the patient or subject at a certain rate set by time intervals. The stimulation unit 644 can be a separate stimulation catheter, for example. Alternatively, in at least one embodiment, the stimulation unit 644 can be part of the pacemaker/ICD unit 160 whether it has a conventional size or is miniaturized.

It will be appreciated that one or more components of the system 600 may be embedded within the pacemaker/ICD unit 160. For example, the pacemaker/ICD unit 160 may have embedded therein the processor unit 604 and the memory unit 618. Meanwhile, other parts of the system 600 may be located on a computing device that is physically separate from the pacemaker/ICD unit 160 but is wirelessly connected to it. These and other variations may be possible.

Referring now to FIG. 7, shown therein is a flow chart of an example embodiment of a method 700 of positioning one of the devices 100, 100a, 100b or 100c for the acquisition and analysis of electrophysiology (EP) data. To simplify the description of the method 700, reference will be made to the device 100. The method 700 can be considered as being performed by at least one processor but to simplify the description of method 700 reference is made to a processor.

At act 710, a medical practitioner, such as a surgeon, fixes the tip 130 of the device 100 within a patient's RA and/or RV heart chamber. The device 100 is connected to a pacemaker/ICD device 160 or in the case of device 100b has a miniature pacemaker/ICD device 160b embedded therein.

At act 720, the pacemaker/ICD unit 160 is used to apply pacing stimuli to the patient's heart.

At act 720, the device 100 senses cardiac signals comprising unipolar voltages through each individual electrodes 120a to 120d.

At act 730, the cardiac signals sensed from each electrode 120a to 120d are sent to an analysis device via the output and the connector 150 of the device 100. For example, the connector may be an IS-4 pin-plug. Prior to sending the sensed cardiac signals to the analysis device, the sensed cardiac signals can be preprocessed by applying amplification and filtering and then digitizing the sensed cardiac signals (i.e., via the data acquisition unit 640). Conventional settings and parameters may be used for the filtering and amplification based on the nature of cardiac signals. Alternatively, in embodiments in which the device comprises at least one processor, such as for a miniaturized pacemaker/ICD unit, the preprocessed cardiac signals are not sent through an output and an output connector but are rather provided to the processor for further analysis. When the digitized sensed signals are provided to a processor, such as a processor of the pacemaker/ICD device 160 or to a processor of another device such as the processing unit 604 of the mapping system 600, the cardiac signals can be processed in a number of different ways depending on the application of the multi-electrode device.

At act 740, the processor then records the sensed signals in a data store such as the database 626. The signal recordal is performed after the signal capture occurs. Here, capture is defined as the moment a train of stimulation produces evoked potentials that are phase-locked with the stimulation (i.e., ability to stimulate the myocardium).

At act 750, an electric field is derived by the processor using the recorded electrophysiological (EP) data that was obtained by the electrodes 120a-120d. A technique for deriving the electric field was previously described.

At act 760, the processor of the pacemaker/ICD device 160, 160b or the system 600 extracts electric field features from the electric field that was derived at act 750. These electric field features can be used by the pacemaker/ICD device 160. The features that can be determined include the electric field span potential (EFSP) and an indication of changes in the direction of the travelling wave and the CV. The EFSP can be used by the pacemaker/ICD device 160 or 160b to make decisions based off a decrement in voltage. The changes in CV and the direction of the travelling wave can be used when the voltage range is within acceptable levels but where there is an irregular change in heart rhythm or it is too erratic or abnormal. The acceptable levels can be variable and may be determined based on experimental or simulation tests. If the pacemaker/ICD device 160, 160b determines that the change in heart rhythm is too erratic or abnormal, the pacemaker/ICD device 160, 160b may then deliver the necessary treatment, which can be preprogrammed depending on the particular situation based on experimental and/or simulation tests. For example, the pacemaker/ICD device 160, 160b may deliver treatment to the heart so the heart is operating within acceptable levels (e.g., by providing a pacing stimulus to the heart). Alternatively, or in addition, the pacemaker/ICD device 160, 160b may provide the results of this analysis (e.g., a determination that the heart rhythm has an irregular change, is erratic or is abnormal) to a processor coupled to the device potentially indicating a need for treatment.

Method 700 advantageously uses the 3D electrode configuration 200 of the device 100, 100a, or 100b. In method 700, after the electric field is determined, the electric field span potential (EFSP) can be derived by calculating the largest Euclidean distance that is formed with the electric field and then scaling this distance with the inter-electrode distance to express the quantity as a voltage. For example, the Euclidean distance is obtained pairwise between the distal tip electrode 120d and each of the three coplanar electrodes 120a-120c to obtain three Euclidean distances. The largest of these three Euclidean distances is then selected and multiplied by the inter-electrode distance for the distance between the two electrodes from which the largest Euclidian distance was obtained to obtain a voltage value which is the Electric

Field Span Potential (EFSP). The voltage value derived using the electric field can act as a surrogate for traditional bipoles in pacemaker/ICD units to use for making decisions. Further, method 700 determines voltage in a more robust way compared to taking raw bipolar voltages with traditional or conventional leads. Traditional leads have electrodes positioned collinearly and are susceptible to the smallest of dislodgements or a change in direction of a traveling conduction wave. Method 700, by using the electric field, can still determine the largest voltage even if a minor lead dislodgment or a change in wave propagation occurs because the electric field can still be rotated and projected onto an axis to find the largest voltage in such cases.

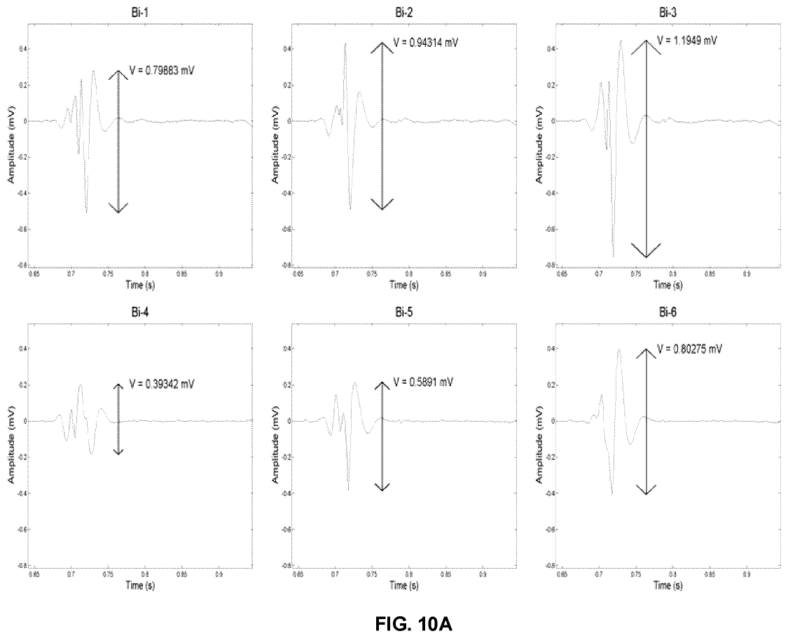

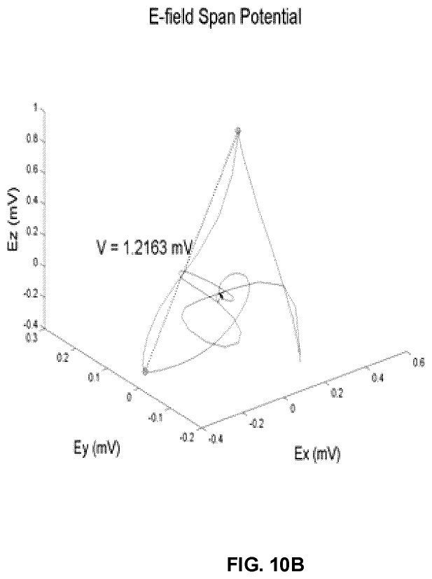

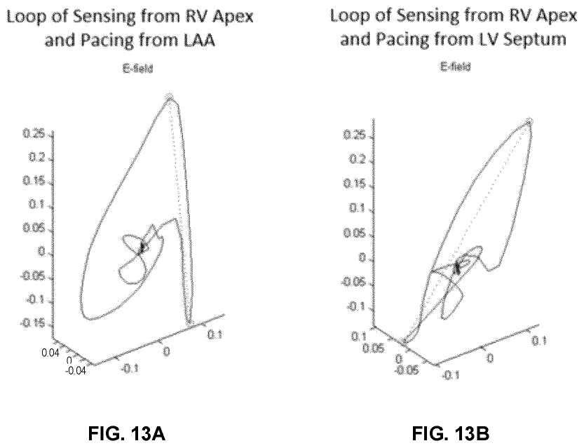

Given the derived 3-D electric field, the quantities that can be used are the EFSP, the geometry (i.e., the geometric profile of the electric field), or a combination of the two. The EFSP can be determined as described above. The geometry can be used as a template to determine changes as explained below. The geometric profile is the outline or loop formed when plotting the 3 electric field vector components. An example of this is shown in FIGS. 10B, 13A, and 13B. The geometric profile that is created when there is no unusual electrical activity is designated as the normal template, and a geometric profile that is created for a new signal is compared to the normal template geometric profile to assess whether the geometric shape of the new signal matches the geometric shape of the normal template geometric profile and therefore whether the newly acquired signal is normal or not.

As the direction of the derived conduction wave (i.e. derived from the determined electric field) is relative to the electrode coordinate system, the determined direction axis (i.e., the direction where the largest voltage is found relative to the origin of the electrode coordinate system) does not correspond to the body's coordinate system. The body's coordinate system aligns with the anatomical position of the person/patient/subject. The electrode coordinate system can be expressed based on an x-, y-, and z-axis where the x and y axis are in a plane that includes the distal tip electrode 120d and is orthogonal with the longitudinal axis of the device 100, 100a, 100b, 100c and the z-axis corresponds to this longitudinal axis. For simplicity, the distal tip electrode 120d can be aligned with the origin while the spatial coordinates of the remaining three electrodes 120a-120c (i.e. vertices) are positioned such that the distance between any two vertices is equal).

The changes in direction of the propagating conduction wave can be used to determine if there are changes in cardiac rhythm. Using the direction axis, which is the projected angle of the electric field that is used to determine the EFSP, the change in angle of the direction axis will suggest a change in the conduction wave source. During the initial calibration or programming stage of the pacemaker/ICD unit 160 or 160b, a medical practitioner, such as a clinician, can sense cardiac signals using the multi-electrode device and determine if the sensed signals are in a normal cardiac rhythm. If the sensed cardiac signals are indicative of a normal cardiac rhythm then the corresponding electric field that can be derived from these sensed cardiac signals, as described previously, can be defined as a normal template (i.e. a template for an electric field corresponding to a normal cardiac rhythm). After the normal template is defined, any change in electric field geometry is determined based on the correlation between the normal template and a given electric field (that is derived after the determination of the normal template from given sensed signals). By normalizing the power of the signals, a dot product can then be taken of the normal template vector and the current vector given that the sample length of the signal to be correlated is the same as the sample length for the template to determine a similarity or matching score between the current electric field and the normal template. The matching score ranges from 1 (i.e. the current electric field has an identical geometry to the normal template) to −1 (i.e. the current electric field has a geometry that is identical but opposite in polarity to the normal template). A matching score threshold can be predefined, for example by the medical practitioner, to identify specific ranges of matching scores that are associated with current electric fields having a geometry which can be classified as being abnormal. The electric fields with an abnormal geometry can then be flagged.

Referring now to FIG. 8, shown therein is a flow chart of an example embodiment of a method 800 for analyzing electrophysiological (EP) data that has been obtained from a device 100, 100a, 100b or 100c having multi-electrodes with a 3D configuration positioned at a distal end of the device. For ease of illustration, the device 100 will generally be referred to in the description of method 800 but it should be understood that the method 800 is also applicable to the other iV multi-electrode devices described herein. The method 800 can be considered as being performed by at least one processor but to simplify the description of method 800 reference is made to a processor.

At act 810, the device 100 measures EP data with its four electrodes. When the electrodes 120a-120d of the device 100 are placed at or within the heart the measured electrophysiology (EP) data represents electrical cardiac activity in the form of electrograms. The electrograms are derived based on the potential difference between any two of the four electrodes 120a-120d.

At act 820, the measured EP data is transferred as an output of the lead device 100, 100a, or 100b through an industry standard pin. This EP data can be transferred to an IS4/DF4 pin plug as each ring on the plug can be separately connected to one of the electrode spheres at the tip of the device 100 or 100c. After being outputted, the measured EP data can be preprocessed by applying amplification and filtering, as described previously for method 700, and then performing digitization. The digitized EP data may then be recorded in a data store such as the database 626, for example, where it can be accessed by a processor such as the processor of the pacemaker/ICD unit 160 or 160b or the processor unit 604 of the mapping system 600.

At act 830, one of the aforementioned processors is used to determine an electric field on a beat-by-beat basis from the digitized EP data that was initially obtained by the four electrodes 120a-120d. From this derived electrical field, the electric field span potential (EFSP) can be determined by taking the product of the maximum Euclidean distance of the field and the inter-electrode spacing as explained previously.

At act 840, the processor generates one or more quantities that are derived from the electric field. These quantities include the 3-D electric field along with its corresponding EFSP, and a binary indicator for the change in conduction velocity and traveling wave direction. The electric field may be stored as 3 vectors where the units are mV/mm (examples of these 3 vectors plotted together are shown in FIGS. 10B, 13A, and 13B). The binary indicator can be determined by comparing a quantity with a fixed threshold or a programmable variable threshold, and if the quantity goes above or below the threshold it will change the binary indicator to show a change has occurred. The binary indicator may be applied to both CV and the traveling wave direction.

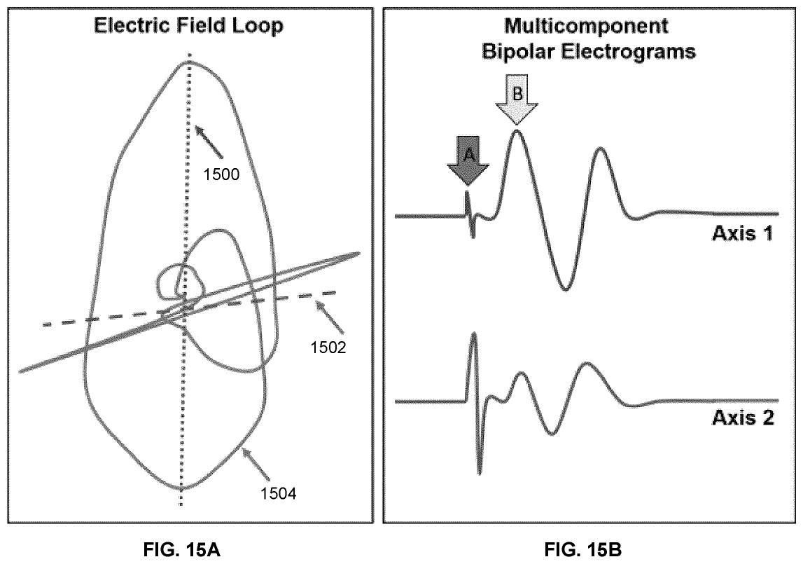

The EFSP serves as the primary source of sensed voltage that the pacemaker/ICD unit uses to determine whether to apply pacing stimuli. An electric field geometry that is obtained during a normal cardiac rhythm, as explained previously, can be used as a template to determine if there are any significant changes to the profile and the direction of the electric field span (i.e., the longest length of the electric field). These changes can serve as a determinant of whether there is a change in direction of the traveling conduction wave, and/or whether a detected arrhythmogenic morphology is still present or similar to the patterns found during a follow-up appointment with a clinician. When His detection is of interest, the electric field can be rotated until the complex associated to His-bundle activity is more dominant in comparison to cardiac muscle activation. The complex associated with His-bundle activity is determined to be more dominant when the largest voltage is found in that respective complex. For example, in at least one embodiment, the rotation is done by taking a projection of the electric field from 0 to 359 degrees on the plane formed by the electrodes 120a-120c (these electrodes may be referred to as the hovering electrodes). This is done assuming the electrode 120d is making proper contact that is reasonably perpendicular to the tissue surface, which reduces mathematical complexity. Although larger step sizes (in terms of degrees between successive projections) will decrease computation time, the projection over some angles may be skipped when the true largest voltage is located. However, at a bare minimum, all angles may be checked. As different activities generate different waves from potentially different sources, the waves will travel past the electrodes and affect the electric field differently. By viewing the electric field at different angles, some activities or complexes may be more emphasized. As an example, FIG. 15A shows red and blue dotted lines that represent 2 different arbitrary angles.

As previously mentioned, conventional leads may face difficulties in detecting voltage in a reliable manner. Hence, misdetection and/or incorrect treatment action provided by pacemaker/ICD units occur as the decision criterion is dependent on the sensed voltage. Although some pacemaker/ICD devices are equipped with better algorithms to deal with unreliable sensed voltages, the initial sensed voltage is still vulnerable to external influences such as noise and lead orientation with conventional leads. As waves travel through different conduction pathways, the orientation of the conventional lead becomes influential as traditional bipolar signals observe the difference in potential signals sensed by the electrodes but this is only in 1 axis without any knowledge of any other axes or any other rotations up to 360 degrees. In contrast, an Electric-field is a 3D structure that can be analyzed from a variety of angles.

In contrast, by using devices with a 3D multi-electrode configuration, in accordance with the teachings herein, reliance on orientation is minimized as many different voltages sensed using the 3D electrode configuration can be used to make different observations as each electrode from the base of the tetrahedral positioning is independent of each other which allows each electrode to sense voltages from the region covered by the tetrahedron differently. With conventional leads, only one electrode is used generally at the position of the four electrodes in the tip of the 3D multi-electrode devices described herein, and using only one electrode results in reduced sensitivity in that localized region. However, in accordance with the teachings herein, better signal quality can be achieved by consistently detecting larger voltages in comparison to the traditional method with conventional leads. For example, every time the voltage needs to be updated, the electric field may be rotated and projected to find the largest voltage.

At act 850, the processor determines a potential difference to represent the electric field based on determining the EFSP and scaling by an inter-electrode distance from the distal end of the lead device 100, 100a, 100b or 100c as explained previously. The electric field has units of mV/mm. For a given 3-D electric field, the span of that electric field is determined by finding the largest Euclidean distance that can be formed. This span represents the maximum bipolar signal but is still expressed in terms of mV/mm. Hence, by scaling the EFSP with the inter-electrode distance, a quantity in terms of potential difference, expressed in units of mV is derived.

At act 860, the processor of the pacemaker/ICD unit 160 uses the electric field span potential (EFSP) as a voltage threshold parameter for the pacemaker/ICD unit 160, 160b to minimize an effect of minor lead dislodgement. For example, at implant the pacemaker/ICD unit 160 may measure 7.5 mV for a traditional bipole, but after the implant the patient may roll over which may make the lead move such that the traditional bipole sensing may drop to 3.5 mV which can cause the pacemaker/ICD unit 160 to not function properly since lower sensing voltage is more similar to, and can be confused with, background noise levels which may be fluctuating around 2 mV, e.g., sometimes higher and sometimes lower. However, the EFSP can minimize the effect of minor lead dislodgement because the electric field is not as dependent on the physical positioning of the electrodes 120a-120d compared to electrodes used in traditional leads to form bipoles. For traditional bipoles, if the 2 electrodes are not aligned properly with the direction of the traveling conduction wave, then the sensed voltage will be minimal or it may not capture the traveling conduction wave. Hence, any changes in traditional or conventional electrode configurations will produce different voltage readings. In contrast, a near identical electric field can be derived in the instance that the proximal electrodes of a 3D multi-electrode configuration, according to the teachings herein, assumed another position on that plane. For instance, referring to FIG. 2B, assuming electrodes 120a-120c rotate on the plane formed by these 3 electrodes, a near identical field may still be formed. The EFSP has the benefit of aligning with the direction of the traveling wave, which enables the EFSP value to be more informative than traditional bipolar voltages by providing direction and speed. In contrast, conventional recorded bipolar voltages may appear to be similar while in actual fact the direction of the conduction wave has changed. For a specific range of values associated with specific treatments or actions, the parameters may be identical to current thresholds predetermined by the pacemaker/ICD unit manufacturers or manually set by the clinician. Since EFSP is less affected by minor lead dislodgement, the EFSP can still be a useful voltage threshold parameter without having to either (a) lower the voltage threshold parameter or (b) readjust the lead to maintain a high enough voltage threshold parameter to have good signal-to-noise ratio (SNR).

At act 870, the processor uses the EFSP as a voltage threshold parameter for the pacemaker/ICD units to determine when these units may apply stimuli to pace or not pace that patient's heart. If the EFSP drops below the threshold, the pacemaker/ICD unit 160 will pace. Since at act 860, the effect of minor lead dislodgement is minimized, in the event of minor lead dislodgement, the processor's use of EFSP as a voltage threshold parameter at act 870 allows the operator to avoid re-performing surgery to reposition the dislodged lead.

For example, in at least one additional embodiment, the voltages sensed by the electrodes 120a-120d of the device 100, 100a, 100b, 100c can be used to detect changes in electric field geometry to distinguish a change or shift in direction of a traveling conduction wave due to different arrhythmogenic sources. For a given stable cardiac rhythm, the electric field geometry generated for each individual beat will be near identical to one another. Taking this electric field geometry as the baseline or reference for a normal template (described previously), a change in the direction of the traveling conduction wave can be detected once the direction of the longest axis of a given electric field and the correlation of the given electric field geometry with the normal template varies. For example, the amount of change in the direction of the traveling conduction wave may conventionally be determined by a clinician or operator who visually determines that the shape of the traveling conduction wave appears to be different. However, in accordance with the teachings herein, a percentage threshold can be applied to the amount of match (i.e. correlation) between the electric field geometry with a template in order to determine the amount of change.

In at least one alternative embodiment of method 800, a processor can detect a first direction of an activated wave by aligning the activated wave with a first longest axis which is a first largest amplitude of a first electric field recorded during normal sinus rhythm. The processor can then compare the first direction with a second direction of a second longest axis which is a second largest amplitude of a second electric field recorded during pace mapping. The processor can then provide a score of how similar the first direction is to the second direction. If the score reaches a predefined threshold, i.e., there is a match, then the next action to be taken depends on whether sensing or pacing is taking place. During sensing, if there is a match (e.g., to the normal template), then nothing needs to be done as the heart has a normal cardiac rhythm, otherwise if there is no match the heart may be experiencing an abnormal cardiac rhythm in which case the pacemaker can be adapted to provide an electrical pulse through the lead to induce the heart to have a normal cardiac rhythm. Alternatively, during pacing, if there is a match (e.g., to the abnormal template) at a particular heart location where the cardiac signals were sensed, then this indicated that this heart location may be the source of an abnormal cardiac rhythm and an ablation catheter may then be used to ablate at this location of the heart that was paced, otherwise if there is no match then an action does not need to be performed. This embodiment is advantageous when using the direction of the activated wave information. For example, when comparing a geometric profile to another geometric profile, it can be computationally demanding to assess whether these geometric profiles match or not for every point on the geometric profile. One possible alternative to comparing each point of the geometric profiles is to only compare the longest axis of a first geometric profile which is its largest amplitude of that first geometric profile with the longest axis of the second geometric profile which is its largest amplitude of that second geometric profile. If the vectors point in the same direction when based in a shared coordinate system, it is an indication that the geometric profiles match each other. Another way to describe the longest axis of the geometric profile is to try to draw the longest line that can be fitted within the loop shape of the geometric profile. That longest line would be considered the longest axis, which is the largest amplitude of that geometric profile.

In at least one additional embodiment of method 800, a processor can determine an electric field and use it to differentiate between changes in conduction velocity. By selecting the axis that produces the largest voltage, the traveling conduction wave will be propagating along that axis. This selection may be done by comparing the voltage values obtained in each iterated projection as described previously. Once found, the angle used to derive the projection is stored. A conduction velocity can then be determined by taking the product between the vector of this axis and the ratio of the peak-to-peak amplitude of the time derivative (of this electrical signal) over the peak-to-peak bipolar voltage. When the heart condition is in a sinus rhythm, the conduction velocity will have minimal variations. However, in the presence of arrhythmia, the derived conduction velocity will be altered, which will cause a change in magnitude and potentially direction of the conduction wave velocity. Accordingly, the presence of arrhythmia can be determined by monitoring whether changes in the magnitude and direction of the conduction velocity are larger than a magnitude and a direction threshold, respectively.

In at least one additional embodiment of method 800, a processor can determine an electric field and use it to maximize His detection by rotating the electric field to emphasize His-Bundle activity while suppressing cardiac muscle activation. By projecting the initially determined electric field at a different angle on the imaging plane that the electrode 120d is on, which is parallel to the plane formed by the electrodes 120a-120c, this results in the electric field being rotated and shows a different aspect of the electrical field components. For example, FIGS. 15A and 15B provide examples of the different electrograms due to the rotation of the electric field to different angles. From the electric field's perspective, each individual signal complex may vary in geometry in response to its frequency characteristics and magnitude. An example of this is shown in FIG. 15A, where there are smooth curves and sharp edges at different locations corresponding to different complexes. Signal complexes mainly contain cardiac activation but other activities (i.e. His-bundle) and/or artefacts may potentially be captured. Furthermore, at certain angles, the rotated electrical signal may align more closely in the direction of certain cardiac activities, which further emphasizes the corresponding complex of that cardiac activity where a complex is the QRS complex. In other words, subtle differences may visually be present when the sensed signals are formed into the electric field and the electric field is viewed from a different perspective based on the projection of the electric field. This is because certain complexes may be more enhanced as the projection is rotated closer to an angle in which the cardiac activity is aligned. For example, if the found angle maximizes the electrogram complex associated to His-bundle activity, His detection can then be performed more easily as the presence of the cardiac muscle activation complex is weakened or attenuated.

Conventional leads are only equipped with electrodes positioned collinearly along the lead's shaft. Consequently, the signal quality is very much dependent on the initial orientation of the lead at the implantation stage. Hence, the differentiation between atrial, His, and ventricular sensed voltage signals is difficult as the ventricular activity is more predominant (i.e. has a larger amplitude) and has a high probability of masking the other complexes. However, using devices with a 3D multi-electrode configuration in accordance with the teachings herein, and the derived electric field as a tool to sense cardiac voltage, provides a better probability of observing the weaker cardiac activities. For example, using devices with the novel 3D electrode configuration provides a means for improved signal quality as it will determine the largest bipolar signal. As another example, the increase in differentiation of cardiac activity may be determined due to the ability to rotate the electric field and based on the axes of the different cardiac signals which are different. As the electric field is rotated, the emphasis of the signal (i.e. the magnitude of the signal complex associating to cardiac activity) will change as the projection of the electric field aligns itself closer with cardiac activities that are along that preferential axis.

Referring now to FIG. 9A, shown therein is a flow chart of an example embodiment of a method 900 of using an electric field measured by a multi-electrode device 100, 100a, or 100b. Method 900 may, for example, be used to obtain the electric field after one or more acts of method 700 and/or method 800 are performed. The method 900 can be considered as being performed by at least one processor but to simplify the description of method 900 reference is made to a processor.

At act 910, a processor, such as the processor of the pacemaker/ICD unit or the system 600, can store an electric field geometry and traits that are identified as being abnormal which can then be used as a template for matching. The term “abnormal” in this context refers to changes in heart rhythm or morphologies deemed different compared to the patient's normal cardiac rhythm. This can be determined as previously described. The abnormal reference or template can then be used to determine whether the abnormality that is detected is similar to an abnormality that is detected by a clinician when sending cardiac signals from a patient when the patient comes in for a check-up. The similarity can be based on determining the matching score, as previously described, and comparing the matching score to a predefined threshold.

At act 920, a clinician performs measurements on a patient using a catheter to introduce a device having a multi-electrode configuration in accordance with the teachings herein. The measured cardiac signals for a given heartbeat can be used by a processor to measure a current electric field for the given heartbeat.

At act 930, the processor determines, based at least in part on a matching score, if the current electric field has a geometry that is similar to the geometry of an abnormal template by comparing the matching score to a predefined threshold. If this determination is true, then various remedial actions can then be taken such as performing ablation or cryofreezing.

For example, in at least one embodiment, when the cardiac signals are recorded during an arrhythmia, the cardiac signals can be sensed by and stored in the pacemaker/ICD unit 160 or 160b and those cardiac signals can be used to create an abnormal electrical field template. Later, during an ablation procedure when an ablating catheter is being moved to different locations in or on the patient's heart, one can activate the pacemaker 160, 160d to record cardiac signals, recreate the electrical field from the sensed cardiac signals to determine if there are conduction wavefronts from the electric fields (as previously described) and if there are conduction wavefronts, then determine a matching score for the electrical field geometry corresponding to these conduction wavefronts originating from these various ablation catheter positions with the abnormal template, thus potentially localizing the source of arrhythmia in the heart when the matching score is above a predefined threshold. If the electrical field geometry produced by an electrical signal from the roving ablation catheter and sensed by the pacemaker 160, 160d matches the abnormal template, it is an indication that the roving catheter is likely located at the same physical location from where the cardiac signals for the recorded arrhythmia originated. Any located arrhythmia sources can then be ablated.

In at least one alternative embodiment of method 900, the processor is configured to record the sensed signals as the EP data after signal capture occurs during an arrhythmia; store an arrhythmia electric field that is derived from the recorded EP data during the arrhythmia; define an abnormal template from a first electric field geometry for the stored arrhythmia electric field that is associated with the arrhythmia; determine a second electric field having a second electric field geometry resulting from pacing at a given heart location during a medical procedure with a roving ablation catheter; determine a matching score by taking a correlation of the first electric field geometry with the second electric field geometry; compare the matching score to a matching score threshold to identify when the second electric field matches the arrhythmia electric field; and when the second electric field matches the arrhythmia electric field, indicate to a medical practitioner that a remedial action be taken comprising ablation or cryofreezing at the given heart location where the pacing by the roving ablation catheter caused the second electric field.

In cardiac pacing, a stimulation threshold may be set which is the lowest amplitude of a stimulation pulse that is needed to cause the heart to depolarize and contract during cardiac pacing. The lower the stimulation threshold, the lower the amount of electrical energy that is used for pacing which results in improved pacing efficiency since less energy is expended by the pacemaker/ICD unit. Typically, a static stimulation threshold is chosen for use and while it is desirable to have a low threshold to save on electrical energy expended for pacing, if the stimulation threshold is chosen to be too low then it may result in ineffective pacing. Furthermore, it may be difficult to choose a lower stimulation threshold since different amounts of energy are used for depolarizing and contracting the heart during pacing due to various factors such as, but not limited to, the position of the electrodes that are used to provide the pacing stimuli, the selections for the anode and cathode used for applying the stimulus threshold, the electrodes being in contact with appropriate cardiac tissue and also other conditions which may affect cardiac tissue that is in contact with the stimulation electrodes where these conditions include, but are not limited to, electrolyte concentration, acidosis and hypoxia.

The inventor has determined that since each of the factors that affect the amount of energy used during pacing depend on electrode position and the anode and cathodes that are used for applying the pacing voltage, the 3D multi-electrode configuration described in accordance with the teachings herein may be used to select the best pair of electrodes for providing a lower stimulation threshold to achieve cardiac pacing with improved pacing efficiency. Furthermore, as described previously, the use of the 3D multi-electrode configuration described herein with the pacemaker/ICD units allow for improved sensitivity in recording the electrical activity that is generated by the heart which will allow the stimulation threshold to be selected more accurately to prevent any competition between the cardiac pacing and the heart's natural cardiac activity.

Referring now to FIG. 9B, shown therein is a flow chart of an example embodiment of a method 950 of providing pacing with lower stimulation thresholds using a multi-electrode device having a 3D electrode configuration and a pacemaker/ICD unit in accordance with the teachings herein to improve pacing efficiency. The pacemaker/ICD unit may have a regular size or may be miniaturized. The method 950 can be considered as being performed by at least one processor but to simplify the description of method 950 reference is made to a processor.

At act 952, voltages are measured by the 3D multi-electrodes. At act 954, an algorithm that is conventionally employed by the pacemaker for determining the pacing stimulus is used to determine the pacing stimulus using the various anode and cathode combinations where the anode and cathode can be selected from any of the electrodes 120a-120d and the housing or an input of the pacemaker that is electrically isolated from the electrodes 120a-120d as long as the anode and cathode are different. Accordingly, there may be 16 different anode/cathode combinations as shown in Table 1 and a pacing stimulus voltage is determined for each anode/cathode combination.

TABLE 1
Anode and Cathode Combinations using 3D multi-electrode device

| Combination | Anode | Cathode |
|---|---|---|
| #1 | Electrode 120a | Electrode 120b |
| #2 | Electrode 120a | Electrode 120c |
| #3 | Electrode 120a | Electrode 120d |
| #4 | Electrode 120a | Pacemaker body |
| #5 | Electrode 120b | Electrode 120a |
| #6 | Electrode 120b | Electrode 120c |
| #7 | Electrode 120b | Electrode 120d |
| #8 | Electrode 120b | Pacemaker body |
| #9 | Electrode 120c | Electrode 120a |
| #10 | Electrode 120c | Electrode 120b |
| #11 | Electrode 120c | Electrode 120d |
| #12 | Electrode 120c | Pacemaker body |
| #13 | Electrode 120d | Electrode 120a |
| #14 | Electrode 120d | Electrode 120b |
| #15 | Electrode 120d | Electrode 120c |
| #16 | Electrode 120d | Pacemaker body |

At act 956, the anode/cathode combination that uses the lowest amplitude pacing stimulus is selected. At act 958, the anode/cathode combination that corresponds to the lowest amplitude pacing stimulus is used to apply pacing stimuli to the patient's heart. For example, this may result in applying a stimulus having an amplitude of 0.5 V for cardiac pacing instead of a stimulus having an amplitude of 5 V if another anode/cathode combination was used for cardiac pacing. The determination of the amplitude of the pacing stimulus for the various anode/cathode combinations can be repeated several times in order to account for statistical variability and select the anode/cathode combination which most consistently provides the lowest amplitude stimulus for cardiac pacing. The selected anode/cathode combination is used until reassessment. This reassessment may be done automatically using software on the pacemaker/ICD unit. This reassessment may be done periodically such as every day, every month or during a follow up which may be every 6 months or once a year.

Referring now to FIG. 10A, shown therein are graphs of example traditional bipolar combination signals (Bi-1 to Bi-6). The graphs plot the amplitude in mV against time in seconds during a heartbeat. Each bipolar signal is obtained by taking the difference in measured potential between two electrodes.

Referring now to FIG. 10B, shown therein is a graph of an example Electric-field span potential (EFSP) for the same heartbeat graphed in FIG. 10A where the EFSP is derived as explained previously. Although the electrodes are in the same position and observe the same cardiac activation, the derived voltage from the EFSP is larger in contrast to all derived bipoles shown in FIG. 10A.

### STUDY 1: Experimental Results

Multipoint sensing has been demonstrated to be superior to conventional bipolar sensing in achieving higher sensing amplitude and diminish far field oversensing.^{1,2} Conventional multipoint sensing uses bipolar electrodes that are disposed along the shaft of the lead and are therefore collinear and do not have a 3D configuration in contrast with the teachings herein. The multipoint sensing lead aids in diagnosing directionality of an electrical impulse and also provides multiple bipolar electrograms to choose from for scar mapping. This knowledge presented an opportunity to incorporate multipoint sensing or Intelligent Voltage (iV) lead technology, in accordance with the teachings herein, in a defibrillator lead. It was postulated that the substitution of a conventional (tip to ring) RV electrode with an iV multi-electrode device, as described herein, can assist in localization of VT via a defibrillator with consequent use in pre-ablation planning. Given this configuration, the electric field can then be derived. Compared to other lead system and methods³⁻⁶, similar electrode configurations and applications were used but not in conjunction with a differently structured electrode placement. This technique can also aid in SVT-VT discrimination in ICDs. The availability of multiple bipoles for sensing can also mitigate the need for RV lead repositioning in the event of R wave undersensing, especially in the pen-implant period.⁷

### Methods

The preliminary study used electrograms extracted from 8 porcine hearts, which was sustained on a Langendorff setup. Porcine heart studies were approved by the Animal Care Committee at Toronto General Hospital (Toronto, ON, Canada). Hearts were harvested from normal, healthy, male Yorkshire pigs and the pigs weighed between 37.4 to 39.5 kg. Signals were recorded at a frequency of at least 1 kHz to capture subtle changes to the electrograms when the pig heart was introduced to an electrical stimulus via pacing. Visual inspection was performed on the detected signals before recording to ensure that the location was detecting mostly the near-field components.

The pacing device used consisted of 2 electrodes with minimal spacing to create a pacing artefact as a single point source. These 2 electrodes were encased into a rigid shaft for controlled maneuvering. With this setup, it ensured proper contact to the tissue surface. The iV device used for sensing consisted of 4 electrodes with identical size, material, and spacing. These electrodes were oriented in a tetrahedral configuration and generated 6 unique bipolar combinations. Bipolar electrograms were formed by taking the signal difference between a unique pair of electrodes. With 4 available electrodes, 6 unique combinations were formed. This was done to simulate different possible lead orientations that may occur when introducing a conventional lead with collinear electrodes. The iV lead electrogram was obtained from the derived electric field using the unipolar signals from each individual electrode and the distance between the electrodes. Based on the tetrahedral configuration formed by the distal and proximal electrodes, it was sufficient to obtain a peak-to-peak voltage by deriving the 3-D electric field (Electric-field).⁸ However, to obtain the voltage of the Electric-field itself, the span of the electric field was taken and scaled with the electrode distance. The maximum voltage representing the Electric-field is denoted as the Electric-field span potential (EFSP), in mV.

### Protocol

Signals were recorded by placing the multi-sensing lead device to the right ventricle and right atrial site of the heart. When the lead device was sensing from the right ventricle, the lead tip of the multi-electrode device was positioned to the apex to minimize movement artefacts in the electrograms. However, the right atrial posed challenges since obtaining proper endocardial access while maintaining the overall functionality of the heart was nearly impossible with the Langendorff setup. As an alternative, the multi-electrode sensing device was positioned on the right atrial surface.

To observe if a pacemaker connected to the multi-electrode device can be used to distinguish wave propagation generated from different locations, the pacing electrode of the pacemaker was positioned on different regions of the heart. In addition to varying pacing sites, the pacing interelectrode distance was also varied to verify if acute changes from the same site can also be segregated. The default pacing artefact in the protocol comprised approximately 4 V with a pulse width of 10 ms delivered continuously at a rate of 1 Hz. Applied voltage and frequency varied between subjects as some had different natural rhythms, and/or were resilient to the stimulation at certain locations of the heart. A marker was placed indicating the pacing artefact if capture was confirmed, which was defined as a cardiac activation being consistently invoked and following immediately after the pacing artefact event.

### Results

### A) Largest Voltage Detected

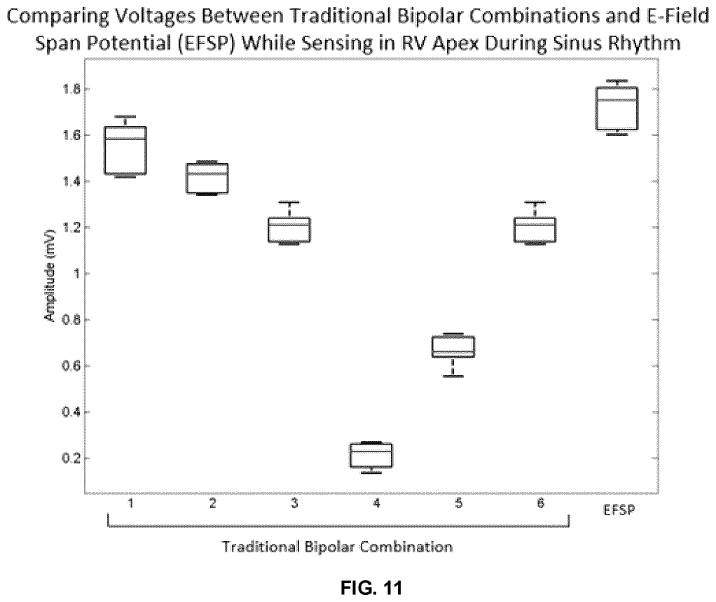

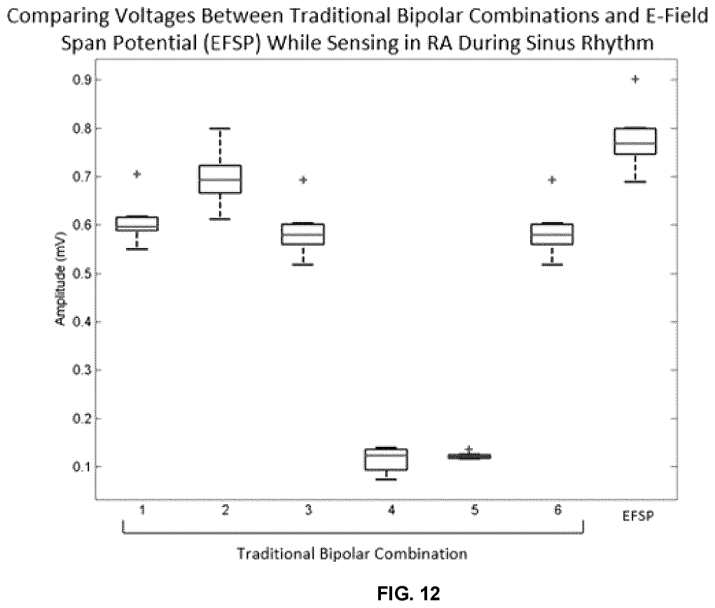

The calculated EFSP appeared to be larger than the voltage obtained using traditional bipolar electrodes over the course of the same 8-11 beats. As shown in FIGS. 11 and 12, the boxplots compare the voltages between traditional bipolar combinations and the EFSP during sinus rhythm while sensing from the RV apex and the RA, respectively. Each combination represents a different bipole orientation on the multi-electrode device. In particular, FIG. 11 shows a boxplot comparing the peak-to-peak voltage between traditional bipoles (combinations 1 to 6) and the EFSP for RV apex sensing during sinus rhythm while FIG. 12 shows a boxplot comparing the peak-to-peak voltage between traditional bipoles (combinations 1 to 6) and the EFSP for RA sensing during sinus rhythm.

According to these results, the EFSP appeared larger than the traditional bipolar voltages and was more consistent in the RA. For the RA sensing site, the 95% confidence interval for comparing the EFSP to traditional bipole combinations 1 to 6 was found to be 0.48 to 1.97, 0.07 to 1.56, 0.18 to 1.67, 1.15 to 2.64, 1.66 to 3.15, and 1.33 to 2.82, respectively.

B) Differentiate Direction

Another finding was that the wavefront originating from different regions can be differentiated. By having the sensing location fixed, the results showed that the Electric-field was different between pacing sites. An example is shown in FIGS. 13A-13B, where the 3-D Electric-field or loop generated by pacing from the LAA (FIG. 13A) and pacing from the LV septum (FIG. 13B) are formed differently. FIGS. 13A-13B show that loops or Electric-fields are different when pacing at different sites while sensing at the same location. In particular, FIG. 13A demonstrates the loop for sensing from the RV apex while pacing from the LAA and FIG. 13B shows the difference in the loop formation when sensing in the same site but paced at the LV septum. As shown by the dotted lines in FIGS. 13A and 13B, the longest axis changed between pacing locations.

### C) Arrhythmia Focus Mapping by Creating a Matching Score

This embodiment deals with an innovative issue of finding the arrhythmic focus without inducing arrhythmia or instances in which arrhythmia cannot be induced at the time of intervention. This is because a pacemaker/ICD that has the tetrahedral electrode configuration has the capacity of recording an electric field at the time of the arrhythmia. The recorded Electric-field during arrhythmia can be used as an abnormal template for matching as described previously. As a clinician is trying to identify where the arrhythmia originated by navigating an ablating catheter in a patient while applying pacing at various locations of the heart from the tip of a non-specific ablation catheter (a conventional electrode), the 3D multi-electrode that is implanted and stationary is recording the Electric-field that is being generated from each of the roving ablation catheter positions which allows the diagnosis to be made away from the pacing sites of the ablation catheter. If the ablating/pacing catheter is in the site where the generated Electric-field matches the Electric-field of an arrhythmia that was previously detected by the 3D multi-electrode device, then one can assume the pacing/ablating electrode is at the site at which the arrhythmia arose and therapy can be delivered there without inducing the arrhythmia to make the diagnosis. The abnormal template Electric-field geometry will match with the catheter's electric field geometry to a degree which is determined from generating the matching score as previously described. The matching score can be used to determine how close the geometry of these electric fields match and will indicate to the clinician that this region should be focused on.

### D) Differentiate CV

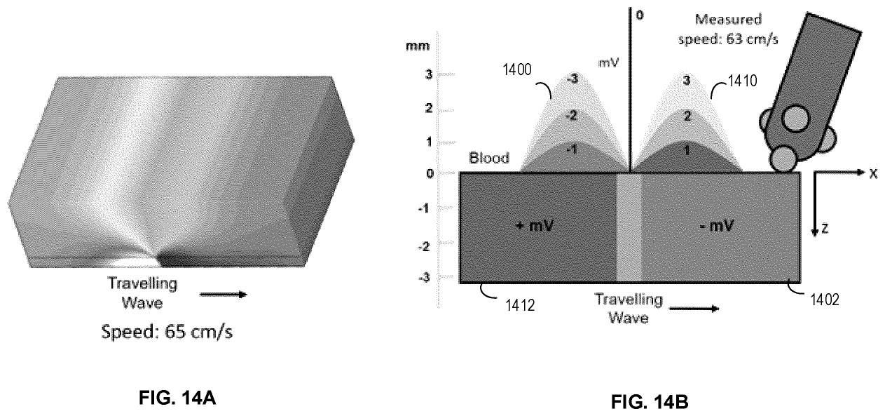

FIGS. 14A-14B show measurement of Wave Speed at the tip of the multi-electrode device. Wave speeds are based on biophysical electric fields that can be measured at the device tip. In particular, FIG. 14A shows a simulation of a wave activation travelling to the right with a speed of 65 cm/s, which is within the range of a normal beating heart. Colors red-to-blue (left-to-right) show the temporal development of the wave. FIG. 14B shows an Electric-field produced by an advancing wave relative to the surface of the myocardium in an in-vivo environment. Colors in red are indicated at 1400 and 1402 and colors in blue are indicated at 1410 and 1412. At the device tip, the ratio of spatial characteristics (i.e. electrode distance) and temporal characteristics (i.e. time derivatives) of the Electric-field can be used to determine the wave speed. This is in contrast to conventional speed determination, which requires multiple local activation times around the area of interest.

### E) Maximize His Detection

Using the iV multi-electrode device and extracting the Electric-field, His-Bundle activity may be better distinguished, as shown in FIGS. 15A-15B. Further examining the bipolar electrograms from the Electric-field, different activity complexes can be seen and differentiated, indicated by the labeled arrows. By identifying a different electrogram component of the Electric-field, the His activity can be further emphasized and become more prominent than the cardiac muscle activity.

In particular, FIG. 15A shows an electric field for Multi-event Detection where the electric field (see the solid-line loop 1504) is produced by a wave activation and can have multiple components within a single event window. These represent unique events, in this case, His-Bundle activation followed by muscle activation. A His-Bundle activation is traditionally characterized by a sharp deflection (see the arrow labeled as A in FIG. 15B) in bipolar electrograms. Following a His-Bundle activation, an interconnected cardiac muscle is activated and is indicated by an immediate following of a relatively sluggish deflection (see the arrow labeled as B in FIG. 15B). Similar to a vectorcardiogram, an electric field can be oriented to give emphasis on individual components within an activation window. Along a first axis (the dotted-line 1500 in FIG. 15A), cardiac muscle activation is highlighted while His-Bundle activity is off-focus. In contrast, along another axis (see the dashed-line 1502 in FIG. 15A), His-Bundle activity is given more emphasis than the cardiac muscle activation. The two axes can be determined when doing the projections of the electric field to obtain the EFSP. The axis 1500 corresponds to axis 1 in FIG. 15B. The axis 1502 corresponds to axis 2 in FIG. 15B.

### Discussion

Placing the iV multi-electrode device into the RA and RV, the EFSP appears to be larger compared to traditional bipolar voltages. It should be noted that the bipoles were generated from the same multi-electrode device and at the same event in time. Hence, the comparisons between the two methods are free from bias. The 6 unique combinations can be treated as a traditional catheter or lead that has the electrodes embedded in a collinear plane at the sensing site at 6 different positions. It is shown in FIGS. 11 and 12 that the sensed voltage varies between bipole combinations although the device tip was fixed at one location. This simulates what the clinician has to face during the implantation of leads. By relying on the Electric-field instead of varying the bipolar lead orientation, the largest voltage reading can still be obtained but with less burden from the clinician. Furthermore, the impact from minor lead dislodgements can be minimized as the calculation of the Electric-field is less dependent on the orientation of the lead thus, making this cardiac signal measurement method more robust.

The electrograms and resultant 3D loops that were derived using potentials sensed by the iV multi-electrode device were consistent when the left ventricular pacing site was unchanged. However, a subtle change in pacing site brought on a significant change in the local iV multi-electrode device electrogram and the corresponding 3-D Electric-field. The sensitivity of the iV multi-electrode in distinguishing distant epicardial pacing site was down to 2.8 mm.

His-Bundle detection appears more distinct while using the iV multi-electrode device. Illustrated in FIGS. 15A-15B are the generated Electric-field and the multicomponent bipolar electrograms. In these bipolar electrograms, specific events can be observed. This feature of the Electric-field can aid to hone-in to and isolate areas of the heart with prominent His-Bundles. This can be useful to improve the procedure and robustness of cardiac device implantation.

### Conclusion of Study #1

The iV multi-electrode devices having the novel 3D electrode configuration described in accordance with the teachings herein can aid in maximizing voltage detection on a lead in an atrium, a ventricle, and for His pacing needs. Processing methodologies that are based on cardiac signals sensed using the iV multi-electrode device leads allow for detecting direction of activation and velocity at the lead tip, which may, advantageously, be used as a mapping tool to detect arrhythmia focus by allowing for reproduction of the arrhythmia while generating a match score during the time the patient is brought in for intervention, i.e. for catheter ablation, when the ablating physician is trying to pace map and reproduce the location of arrhythmic focus such that ablative therapy can be delivered. It should be noted that the ablation catheter is a separate catheter that is used to rove the heart and apply pacing while the stationary 3D multi-electrode is recording the sensed cardiac signals which are used to try to match the measured electrograms to various positions from which an activation is started. When the electrogram matches the previously recorded arrhythmia electrogram the pacing site must be at the focus and thus this is an innovative way of identifying the focus even when the patient is not in arrhythmia. The 3D multi-electrode device with an IS-4 industry standard pin-plug is easily incorporated into current day pulse generators.

### STUDY 2: Experimental Results

Note: experimental results from this second study refer to a multi-axes lead which the reader should understand to be the same device as the aforementioned 3D multi-electrode device.

Changes in sensing and/or pacing threshold after pacemaker implantation is a clinical problem frequently necessitating lead revision, especially during His bundle pacing. SVT-VT discrimination and VT chamber localization is currently not possible by device interrogation.

The aims of this second study were to test if the Electric Field Span Potential (EFSP) and direction specific Electric-field generated from multiple electrodes of a multi-axes lead may record higher sensed voltage and aid in identifying the direction of wavefront respectively and also to test if low pacing thresholds of some bipoles may compensate for high thresholds in others in myocardial and conduction system pacing.

Multi-axes sensing (MS) was demonstrated to be superior to conventional bipolar sensing in achieving higher sensing amplitude and in mitigating far field over sensing.^{1,2} MS assists in ascertaining the direction of wavefront and in addition provides multiple sensed electrograms for analysis. Multi-axes pacing (MP) may ensure that lead micro-dislodgement may be managed conservatively as one or more of the bipoles may provide acceptable thresholds. This knowledge presented an opportunity to incorporate MS and MP in cardiac implantable device (CIED) lead.

By virtue of the tetrahedral configuration of MS electrodes, a direction sensitive electric field (Electric-field) loop may be derived. This may assist in the gross localization of VT and also in SVT-VT discrimination. The availability of multiple electrodes for sensing and pacing may mitigate the need for RV lead repositioning in the event of under sensing or rising pacing threshold, especially in the immediate post-operative period. This is particularly relevant in physiological pacing, especially His bundle pacing, which is plagued by significant pacing threshold increases in the short and long term.⁹

### Methods

Electrograms were recorded from seven swine and two rabbit hearts (to observe sensing and pacing capabilities, respectively), which were sustained on an ex vivo beating heart Langendorff setup, the details of which were described by Si et al., 2018).¹⁰

For sensing experiments, the hearts were harvested from normal, healthy, male Yorkshire pigs weighing 37-39 kg. Signals were recorded at 1 kHz, to capture subtle changes to the electrograms during pacing. Hearts used for pacing were harvested from normal, healthy, male New Zealand white rabbits weighing 3-5 kg and 37-40 kg sheep. Seven fresh sheep heart slabs containing clearly identifiable proximal left bundle (LB) were studied to assess conduction system pacing. The pacing and sensing capabilities were investigated, each with their respective experimental setup. The Animal Care Committee at Toronto General Hospital approved this study.

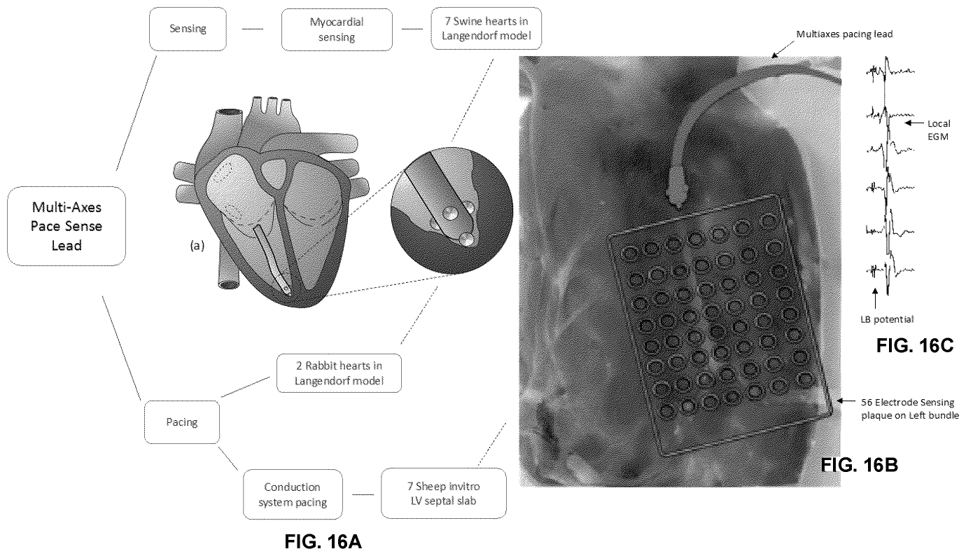

The study setup is illustrated in FIGS. 16A to 16C. FIG. 16A shows that a multi-axes lead was deployed in the apex of right ventricle. Sensing was performed from this multi axes electrode during SR, LV pacing and VF. FIG. 16B shows a left bundle exposed in a fresh sheep LV septal slab. Pacing was performed from the proximal end of the left bundle branch, and conduction system capture was verified by recording from the 56-electrode plaque placed on the left bundle. Capture threshold was assessed by pacing from various bipoles of the multi-axes catheter. FIG. 16C shows conduction system capture proved by the presence of left bundle potential preceding the local EGM.

The aforementioned sensing and pacing was repeated with retraction of a lead 3 mm from the apex thereby simulating lead dislodgment. Conduction system pacing was simulated in seven fresh sheep hearts in a saline bath.

### Multi-Axes Sensing

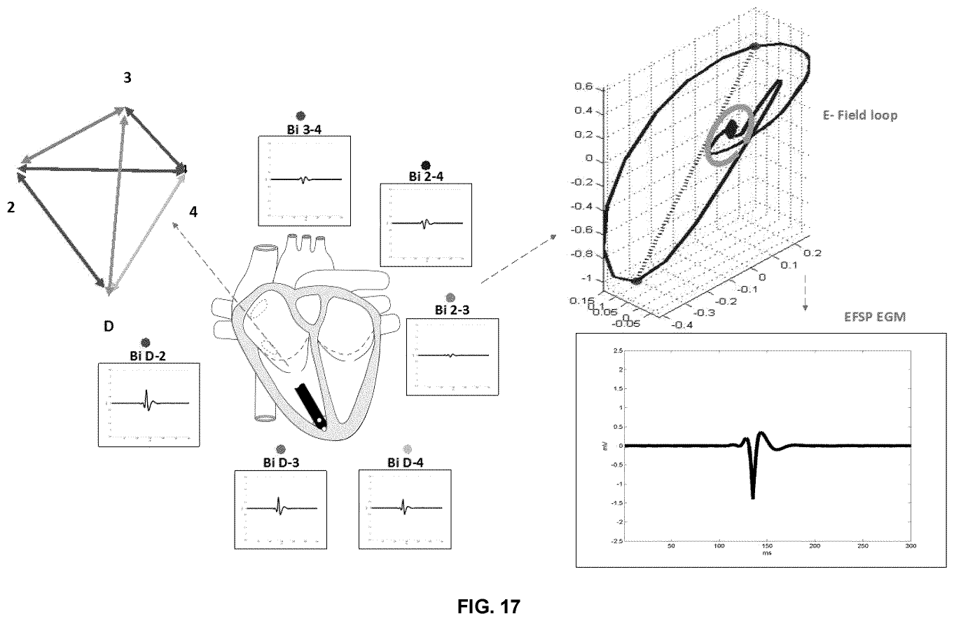

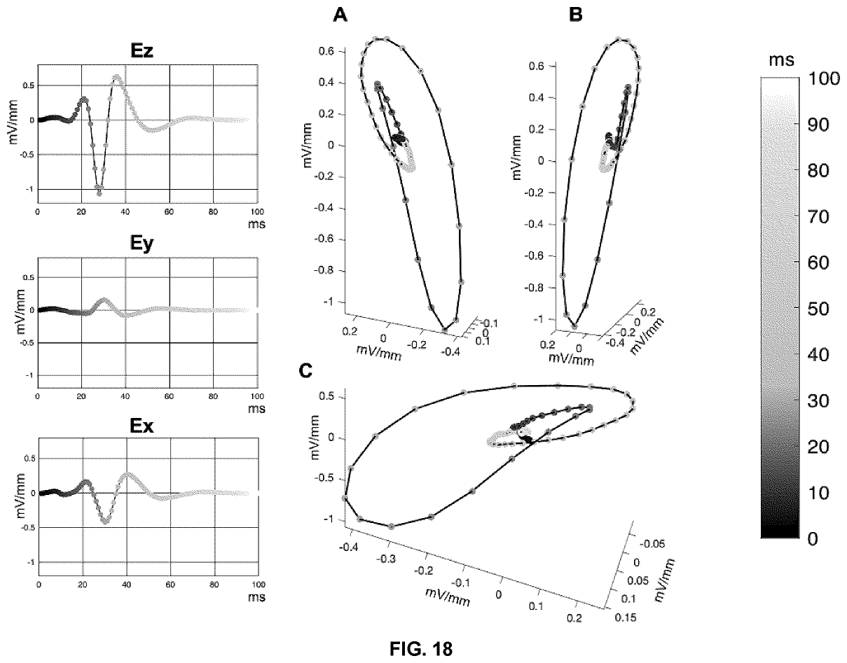

The first feature that was explored was the sensing capability of the multi-axes lead. The pacing probe consisted of two electrodes with minimal spacing to create the pacing artefact as a single point source. These two electrodes were encased into a rigid shaft for controlled maneuvering, which ensured proper contact with the tissue surface. The multi-axes lead used for sensing consisted of four electrodes of identical size, material, and spacing. The multi-axes lead used is illustrated in FIG. 17, which shows how, from the multi-axes lead, the resulting six sensed bipolar electrograms were used to form the 3-D Electric-field loop. By rotating the field, the maximum span was found. The projection of the field on this axis will result in the EFSP EGM. The electrodes were oriented in a tetrahedral configuration that generated six unique bipolar combinations. The electrode diameter used was 1 mm with an interelectrode spacing of 2.8 mm. The multi-axes lead electrogram was derived from the six bipolar electrograms. Based on the tetrahedral configuration formed by the distal and proximal electrodes, it was sufficient to obtain a peak-to-peak voltage by deriving the 3-D electric field (Electric-field), which is expressed in mV/mm. To derive this Electric-field, a set of bipolar signals was derived using the amplitude difference between each pair of unipolar signals. It was assumed that the overall set of electrodes was not positioned collinearly. Using the set of unique electrode pairs and their corresponding physical displacement coordinates, a spatial displacement matrix was then formed. The displacement coordinates are organized with respect to the lead's x-, y-, z-coordinate system. By using the negative product between the set of derived bipoles and the inverse spatial displacement matrix, the Electric-field was determined. To obtain the voltage of the Electric-field, the span of the Electric-field was taken and scaled with the electrode distance. The maximum voltage representing the Electric-field is denoted as the Electric-field span potential (EFSP), in mV. This process of deriving the EFSP from the sensed bipoles is illustrated in FIG. 17. To demonstrate the 3-D aspect of this Electric-field, different rotations of the field are shown in FIG. 18. In particular, FIG. 18 shows that the multi-axes lead uses the six sensed bipolar electrograms to derive the Electric-field vector components. From these vectors (x, y, z), the 3-D Electric-field loop may then be formed. Each time point, in ms, was marked in which grayscale color indicates the time points—with darkest gray being early and lightest gray being late. Different angles of the same Electric-field loop have been shown in images A, B, and C.

Ventricular activation wavefront was recorded by placing the multi-axes lead at the RV apex. To observe if the device was able to distinguish the wave propagation generated from different locations, the pacing was performed from different chambers. The default pacing comprised of 4 V pulse with a pulse width of 10 ms delivered continuously at a rate of 60 pulses per minute yielding a heart rate of 60 heart beats per minute (BPM). Applied voltage and frequency varied between subjects, as the sinus rates were different. Electrogram recordings were only taken for analysis when capture had been established.

To determine if the EFSP was consistent, peak-to-peak voltage was derived from all unique bipole combinations and from the electric field over the course of 8-11 beats. Given the sensing site at the RV apex, the pacing site was varied between the LV and RV chamber. Additionally, the sensing was also performed during sinus rhythm and ventricular fibrillation.

### Multi-Axes Pacing

Two animals were studied for post lead dislodgment pace threshold. Pacing capabilities of the multi-axes lead was explored by placing the device in the RV apex of two rabbit hearts and delivering electrical stimulations in unipolar and bipolar configurations. The unipolar pacing was configured using electrodes of the multi-axes lead and a clip at the base of the heart. For bipolar pacing, two of the four electrodes were paired and alternated until all six unique combinations were achieved. A parameter to measure the pacing capability of the multi-axes lead, the minimum voltage required to induce capture, also known as the voltage threshold, was recorded. The pulse duration was fixed at 0.5 ms to emulate standard pacemaker unit settings. The delivery rate was adjusted between 2 to 3 Hz as each subject had a different intrinsic heart rate. After recording the voltage threshold for each electrode pair in unipolar and bipolar configuration, the multi-axes lead was vertically withdrawn 3 mm to simulate lead micro-dislodgement. All pacing configuration recordings were repeated to confirm changes in the minimal voltage threshold.

### Conduction System Pacing

Seven fresh sheep heart slabs containing a clearly identifiable proximal left bundle were studied, as shown in FIGS. 16A to 16C. Left ventricular slabs were cut out to observe the conductive system and muscle activity in a controlled environment. Each slab was removed from the sheep heart and placed in a well with circulating Tyrode's solution. Bipolar and unipolar pacing stimuli were delivered to the slab through the multi-axes lead electrodes at the proximal end of the conduction system. The propagating EGM was recorded using a 56-electrode custom-made plaque¹¹. The pacing width (0.25 ms & 0.5 ms) and amplitude (V) were varied. A custom mapping system¹² was used to collect all data and determine capture online. The conduction system capture was confirmed by the local electrogram characteristics, the presence of the Purkinje potential preceding the QRS and the speed of conduction. This was differentiated from muscle capture by demonstrating lack of tissue capture by directly pacing the muscle at physiological outputs. However, the muscle may be captured at very high pacing outputs (>20V).

### Statistics:

A hierarchical mixed effects linear regression model was used to compare the maximum bipole and EFSP measurements in order to account for repeat measurements. The nested model included two levels, accounting for multiple measurements within each heart and for the different rhythm and pacing methods in the various heart chambers. For comparisons of pre and post dislodgement, a simpler model with a single random effects variable, accounting for multiple measurements within each heart, was used.

### Results

### Sensing

### (a) Multi-Axes Sensing Voltage (EFSP) was Higher Than Conventional Bipolar Voltage

The calculated EFSP from six bipolar EGMs was larger than the voltage obtained from any traditional bipoles over the course of 8-11 beats. This is illustrated in FIG. 17. The average EFSP shows larger peak-to-peak voltage values in comparison to the individual average bipolar voltages as shown in Table 2. Regardless of the origin of the pacing site or sinus rhythm, the EFSP consistently obtained larger voltages compared to the individual bipolar values obtained.

TABLE 2
Summary of average peak-to-peak sensed voltage obtained
by the unique bipole combination and electric field

| | | | | | | P Value |
|---|---|---|---|---|---|---|
| | | Lowest | Mean | Highest | | EFSP vs. |
| Sensing | Pig | Bipolar | Bipolar | Bipolar | | Highest |
| RV | Serial | Voltage | Voltage | Voltage | EFSP | bipolar |
| Apex | No. | (mV) | (mV) | (mV) | (mV) | amplitude |
| Sinus | 1 | 0.2 ± 0.05 | 0.9 ± 0.5 | 1.5 ± 0.1 | 1.7 ± 0.1 | NS |
| Rhythm | 2 | 0.05 ± 0 | 0.1 ± 0.1 | 0.2 ± 0 | 0.2 ± 0 | S |
| | 3 | 0.07 ± 0 | 0.1 ± 0 | 0.2 ± 0 | 0.2 ± 0 | S |
| | 4 | 0.02 ± 0 | 0.02 ± 0 | 0.03 ± 0 | 0.1 ± 0 | S |
| | 5 | 0.7 ± 0 | 1.1 ± 0.3 | 1.5 ± 0 | 1.6 ± 0 | NS |
| RV | 1 | 0.5 ± 0.1 | 1.3 ± 0.6 | 2.2 ± 0.3 | 2.3 ± 0.3 | S |
| pacing | 5 | 0.5 ± 0.3 | 1.1 ± 0.5 | 1.8 ± 0.1 | 1.9 ± 0.1 | S |
| | 6 | 0.5 ± 0.2 | 1.9 ± 0.8 | 2.8 ± 0.4 | 3.2 ± 0.2 | S |
| | 7 | 0.5 ± 0.0 | 1.5 ± 0.6 | 2.1 ± 0.5 | 2.3 ± 0.2 | S |
| LV | 1 | 0.4 ± 0.1 | 0.8 ± 0.3 | 1.3 ± 0.1 | 1.4 ± 0.1 | NS |
| pacing | 2 | 0.6 ± 0.1 | 2.6 ± 0.6 | 4.2 ± 0.5 | 4.9 ± 0.4 | S |
| | 3 | 0.1 ± 0 | 0.2 ± 0.1 | 0.3 ± 0.0 | 0.4 ± 0 | S |
| | 4 | 0.02 ± 0 | 0.03 ± 0 | 0.1 ± 0.01 | 0.1 ± 0.02 | S |
| | 5 | 1.7 ± 0.03 | 3.1 ± 1.5 | 4.9 ± 0.1 | 5.4 ± 0.1 | S |
| VF | 5 | 0.6 ± 0.2 | 1.2 ± 0.7 | 1.9 ± 0.7 | 2.4 ± 0.7 | S |
| | 7 | 0.5 ± 0.2 | 0.9 ± 0.4 | 1.3 ± 0.3 | 1.7 ± 0.5 | NS |

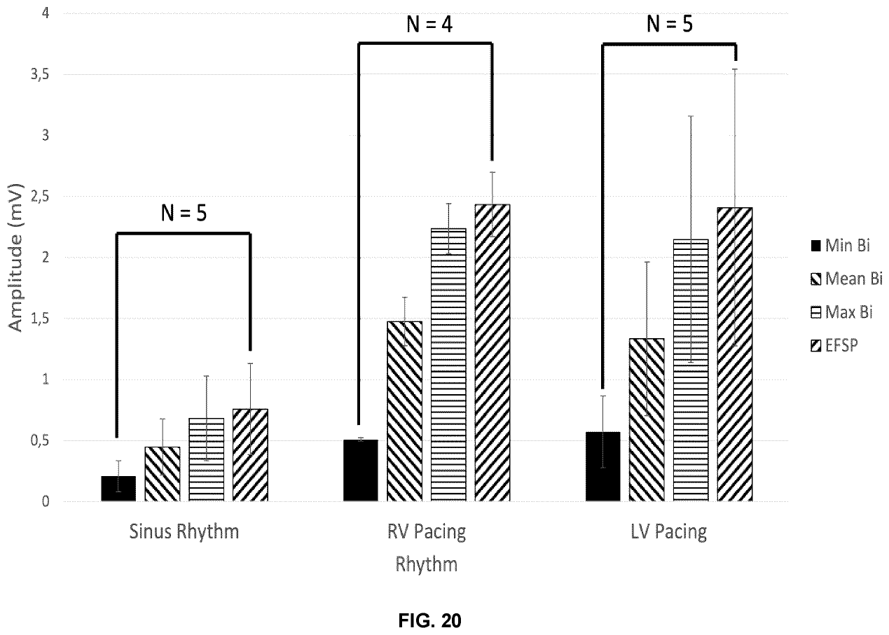

The detected voltage using EFSP was significantly different compared to the maximum individual bipolar voltages. The voltages were compared in three different rhythms (sinus, LV pacing, and RV pacing). Using a mixed model analysis, the voltage difference between max bipoles and EFSP was −0.1757 mV, 95% CI (−0.2787 to −0.0727), p value—0.001, as shown in FIG. 20. FIG. 20 illustrates a comparison of minimum, mean and maximum voltage of bipolar configuration and EFSP during different rhythms. EFSP voltage was consistently larger compared to any bipole. The absolute values are tabulated in Table 2. Regardless of rhythm, EFSP detected larger voltage compared to the maximum bipole.

(b) Multi-Axes Sensing Electric-Field Loop Morphology Changed when Paced from Different Locations which Aids in Determining Directional of Wavefront

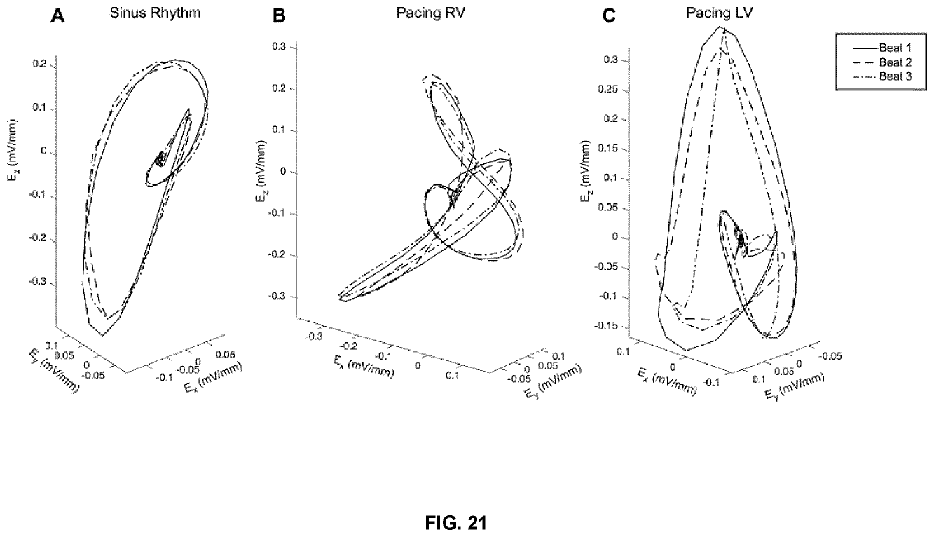

By keeping the sensing location fixed, the wavefront originating from different regions of the heart may be differentiated. The sensed Electric-field loop morphology was different between pacing sites. FIG. 21 shows the Electric-field loops generated for three beats during sinus rhythm (image A), RV pacing (image B), a LV pacing (image C). By changing the pacing site, the loop profile changed and was visibly different in comparison to the loops during sinus rhythm. As a result, this altered the longest axis and EFSP electrogram. In essence, this corresponded to a distortion to the loop itself. By selecting the sinus rhythm beat to be the reference, a correlation coefficient between the other rhythm conditions were taken to numerically quantify the similarity. For loops formed in RV pacing and LV pacing, the correlation coefficient in comparison to the loop from sinus rhythm were 0.31±0.16, and −0.33±0.09, respectively.

(c) Multi-Axes Sensed 3-D Electric-Field Loop is Reproducible

In the ideal scenario, each Electric-field loop will be congruent to the next if the pacing and sensing sites were stable and the recording is free of noise artefacts. Comparing between the 3 beats within each condition, the loops appeared visually similar. To ensure that the EFSP values were reliable, the stability of the field was observed, as shown in FIG. 21. By selecting a reference or template beat for each rhythm, a correlation coefficient was taken to numerically quantify the similarity. For sinus rhythm, RV pacing, and LV pacing, the correlation coefficients in comparison to their corresponding reference beat were 0.97±0.03, 0.99±0.01, and 0.91±0.02, respectively.

### Pacing

(a) Multi-Axes Pacing from RV Apex Demonstrated Significant Difference in Capture Threshold Between Bipoles

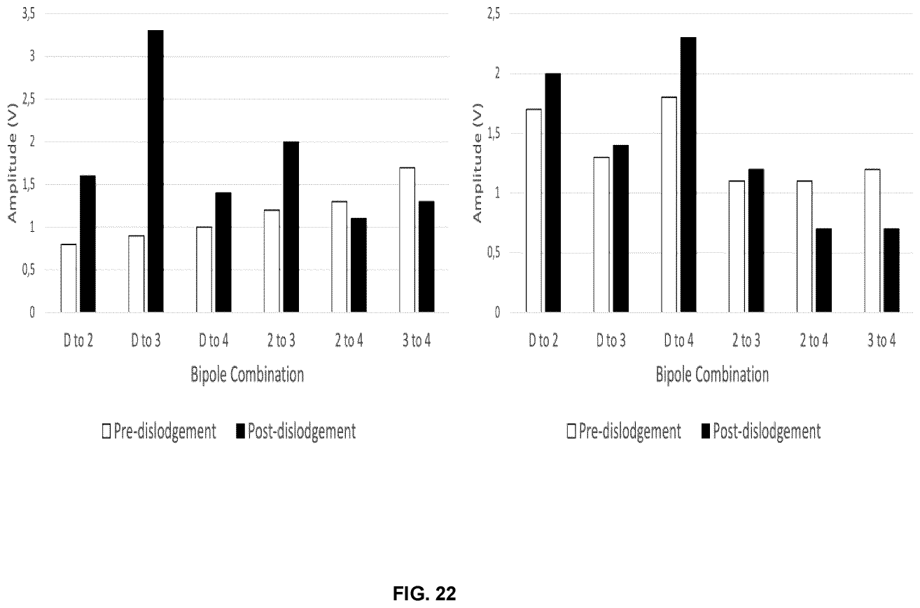

Pacing was performed via the multi-axes lead positioned at the RV apex. Absolute voltage thresholds and relative changes during unipolar and bipolar pacing configurations are summarized in FIG. 22. FIG. 22 shows multi-axes pacing at the RV apex, and in particular a comparison of voltage threshold in two animals. Within each graph, the voltage threshold for each bipole configuration was examined pre- and post-dislodgement. The difference in voltage threshold before and after lead dislodgement was proven not to be significant.

The variability of the pacing voltage threshold can also be observed in FIG. 22. Given the available electrode pairs from the multi-axes, the required voltage for capture varies between electrode pairs. This was especially seen after a lead micro-dislodgement, where the voltage threshold changed for the same electrode pairing. The difference in voltage threshold between the before and after dislodgement conditions was found to be 0.2 mV, 95% CI (−0.5132, 0.9132), which was not statistically significant. The increase in voltages in some bipoles was offset by lower voltages in others.

### (b) Multi-Axes Pacing on the Conduction System Demonstrated Significant Difference in Capture Thresholds

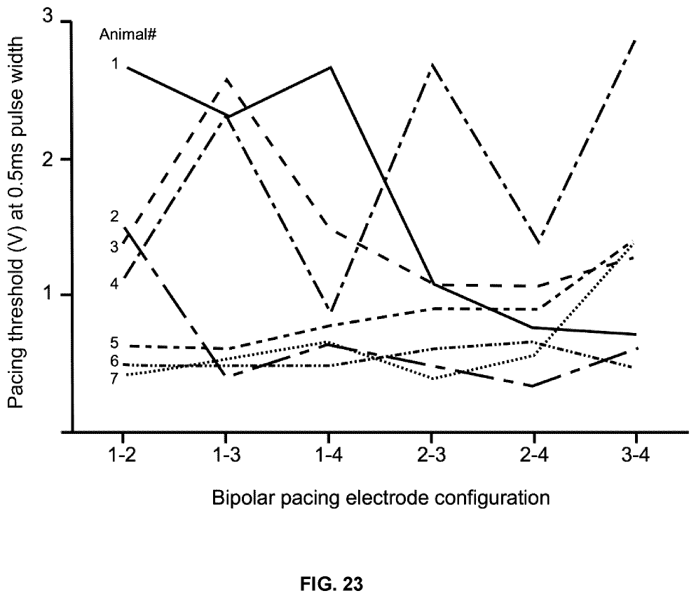

Multi-axes pacing from different bipoles were performed and pacing threshold documented, as illustrated by the setup shown in FIG. 16B. The voltage threshold was assessed in two pulse widths of 0.25 and 0.5 ms as they have the most clinical significance. There was a significant difference in threshold between different bipoles, probably indicating the variation in the direction of bipoles influencing capture threshold, as shown in FIG. 23, which shows left bundle pacing threshold measured from different bipoles of the multi-axes lead in 7 different animals. In all animals, at least one bipole threshold was less than 1 mV and 5 of 7 had one bipole with a threshold less than 0.5 mV. In animals with a high threshold (>2 mV) in one or more bipoles, two to three bipoles in each animal had thresholds less than 1 mV.

### Discussion

The second study showed that EFSP voltage obtained from the multi-axes lead was higher than the voltage recorded from individual conventional lead bipoles in sinus rhythm, ventricular pacing and also in ventricular fibrillation. The three dimensional Electric-field loop derived from multi-axes sensing at the RV apex was reproducible when paced from a distant fixed site, and the morphology of the loop differed when the pacing site was changed. The collective multi-axes pacing thresholds were not statistically significant after deliberate lead micro dislodgement. During conduction system pacing, the high thresholds of some bipoles may be compensated by the low thresholds of other bipoles.

### Utility of Multi-Axes Lead in Defibrillators

The Electric-field loops obtained from the RV apex were consistent in morphology when the ventricular pacing site was unchanged, while the change in pacing site (chamber) brought about a significant change in morphology and direction. This has potential use in SVT-VT discrimination in defibrillators, if one may store the sinus, SVT and VT Electric-field loops. This may then be compared against the rhythm in question. When a device is implanted in a patient and the patient is in a regular normal rhythm, the activation of this at the tip of the multi axes electrode is recorded and a template is constructed. This template will be the reference template. What this implies is that any deviation from this template suggests that the rhythm of the patient is no longer a normal rhythm and is abnormal. It will help differentiate, not only normal from abnormal, but also the location from where the abnormal rhythms are originating. When a template is constructed and does not match the normal template, that recording is saved in computer memory as the abnormal Electric-field loop. The Electric-field loop may also assist in ascertaining the VT chamber of origin by pacing with a roving catheter throughout the myocardial chambers, pacing from various locations, and creating Electric-field loops for each of those pacing locations until the resulting Electric-field loop matches the recorded abnormal Electric-field loop. This allows for identifying the location from which the abnormal rhythm originated, allowing for it to potentially be ablated. This has potential use in pre-ablation planning and also in assessing the success of ablation of a particular VT. The EFSP voltage obtained from the multi-axes lead was significantly larger than the voltage recorded from individual conventional lead bipoles. This may help in resolving the issue of R wave under-sensing during sinus rhythm and also in VF.

### Utility of Multi-Axes Lead in Pacemakers

As expected there was a change in the capture threshold in individual electrodes due to micro-dislodgement. This implies that the pacing voltage will not remain constant and will drain more power from the ICD/pacemaker unit to maintain capture. However, using the multi-axes, this effect may be minimized as more electrodes for use in pacing are available. By programming the ICD/pacemaker unit to select the electrode that delivers the lowest capture threshold, energy consumption from the pacemaker unit will be reduced. In pre-specified programmable time intervals, the ICD/pacemaker unit will assess which of the electrode configurations has the lowest capture threshold and use that configuration, thereby saving drainage of power. This is in contrast to traditional ICD/pacemaker systems with bi-polar leads which only use one single electrode configuration and therefore have no choice but to use a single threshold (which includes a large safety margin which is often 2 or 3 times higher than it has to be). This high threshold leads to drainage of power since it requires higher voltage from the ICD/pacemaker unit's battery.

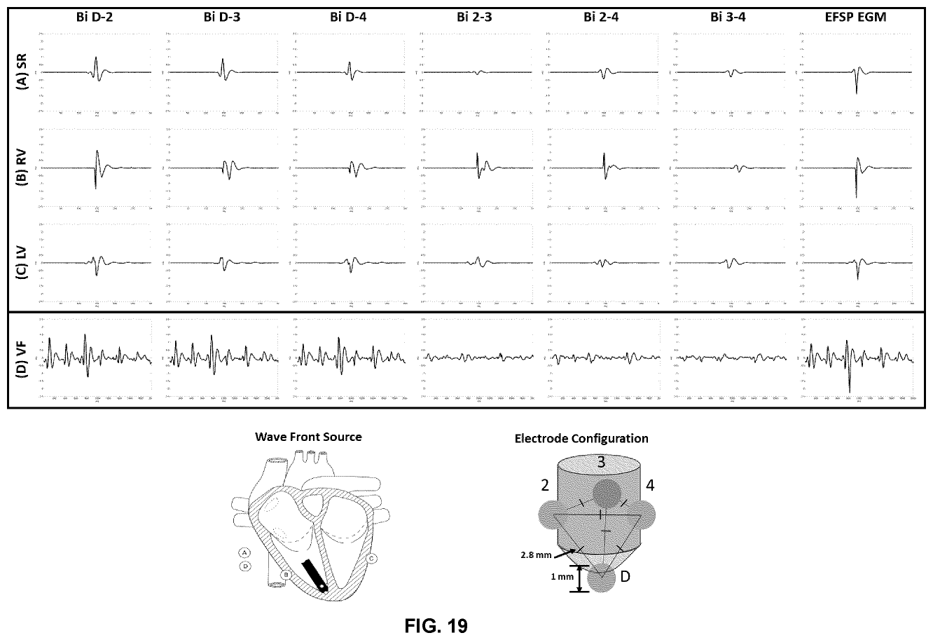

The multi-axes lead EFSP voltage was consistently higher compared to the conventional bipolar voltages. It should be noted that the bipolar and EFSP voltages were generated simultaneously from the same ventricular activation. The six unique bipolar combinations may be considered to operate in the same manner as a conventional lead that has the electrodes embedded in a collinear plane at the sensing site from six different positions. FIG. 19 illustrates an experimental setup and electrode configuration used to observe the difference in sensed voltages between the bipolar combinations and the EFSP. The sensing multi-axes lead was placed at the RV apex. The labeled sites A, B, C, and D, correspond to the electrograms created during sinus rhythm, pacing from the right ventricle, pacing from the left ventricle, and ventricular fibrillation, respectively. It is shown in FIG. 19 that the sensed voltage varies between bipole combinations even though the sensing lead position was unchanged. This simulates the clinical scenario during lead implantation. By relying on EFSP the largest voltage may still be obtained without lead repositioning after implant.

### Utility of Multi-Axes Lead in His Bundle Pacing

The field of physiological pacing has advanced significantly over the last couple of years. This has shown promise not only as a substitute for cardiac resynchronization therapy but also for bradycardia pacing. However the major limitation of His bundle pacing is chronic increase in the pacing threshold.⁹ However, multi-axes pacing may provide a solution for this pressing concern. A problem with a conventional single bipole lead is that it is significantly affected by the angle in which is placed at a location of the heart. Therefore, different angles at a heart location may result in different sensed voltages. This is problematic since the resulting sensed voltage may then be ‘set’ by the surgeon as the voltage threshold parameter. Therefore, when implanting a conventional lead, the surgeon will twist/turn the bipole until the surgeon sees which angle leads to the largest sensed voltage because a larger sensed voltage can more easily be differentiated from noise and therefore can more reliably be used as the voltage threshold parameter. In contrast, the subject multi-electrode device of the present teachings has several bipoles (with 6 pair combinations) so even if one pair is not positioned in a way that leads to large sensed voltage, another electrode pair may be aligned well enough, so that the EFSP that results from the sensed voltage is different enough from the noise that it provides a good voltage threshold parameter. Therefore, with the multi-electrode device of the present teachings herein a suboptimal angle in one pair of electrodes may be countered by a better angle in another pair of electrodes. In fact, this study shows that pacing the conduction system using different bipoles at different angles may influence the threshold. Reasonable thresholds in some bipoles may mitigate the increase in threshold in one or more other bipoles. The variation in threshold from different bipoles may be due to the difference in the contact of these electrodes to the tissue surface.

### Conclusion of Study #2

The multi-axes technology in implantable leads provides the unique ability of maximizing sensing and pacing capabilities on an implantable cardiac device. It also facilitates detection of chamber of the source of activation with potential use in SVT-VT discrimination and VT localization. Compatible with the IS-4 industry standard pin-plug, the multi-axes technology described herein may be easily incorporated into current day pulse generators.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

1. Gerstenfeld E P, Sahakian A V, Baerman J M, Ropella K M and Swiryn S. Detection of changes in atrial endocardial activation with use of an orthogonal catheter. Journal of the American College of Cardiology. 1991; 18: 1034-42.

2. GOLDREYER B N, OLIVE A L, LESLIE J, CANNOM D S and WYMAN M G. A new orthogonal lead for P synchronous pacing. Pacing and Clinical Electrophysiology. 1981; 4: 638-44.

3. Thakur P, Maskara B, Shuros A, Saha S, and Shome S. Augmented signal vector analysis to suppress global activation during electrophysiology mapping. US20130345577A1. 2013.

4. Edwards J, Flickinger W, and Jascob B. System and method for detecting dislodgement of an implantable medical device. U.S. Pat. No. 6,807,439B2. 2001.

5. Costello B, Robertson T, Zdeblick M, Savage G, Colliou O, and Thompson T. Electric tomography. US20080058656A1. 2008.

6. O'Neill. Formable cardiac pacer lead and method of assembly and attachment to a body organ. U.S. Pat. No. 4,154,247A. 1979.

7. Lee D S, Krahn A D, Healey J S, Birnie D, Crystal E, Dorian P, Simpson C S, Khaykin Y, Cameron D, Janmohamed A, Yee R, Austin P C, Chen Z, Hardy J and Tu J V. Evaluation of Early Complications Related to De Novo Cardioverter

Defibrillator Implantation: Insights From the Ontario ICD Database. Journal of the American College of Cardiology. 2010; 55: 774-782.

8. Deno D C, Morgan D, Bush J, Nanthakumar K, Masse S, and Magtibay K. Orientation Independent Sensing, Mapping, Interface and Analysis Systems and Methods. US20180296111A1. 2018.

9. Vijayaraman P, Naperkowski A, Subzposh F A, et al. Permanent His-bundle pacing: long-term lead performance and clinical outcomes. Heart Rhythm. 2018; 15(5):696-702.

10. Si D, Azam M A, Lai P F, et al. Essential role of ryanodine receptor 2 phosphorylation in the effect of azumolene on ventricular arrhythmia vulnerability in a rabbit heart model. Journal of cardiovascular electrophysiology. 2018; 29(12):1707-1715.

11. Haldar H K et al., “Resolving Bipolar Electrogram Voltages During Atrial Fibrillation Using Omnipolar Mapping Circulation: Arrhythmia and Electrophysiology” https://www.ahajournals.org/doi/10.1161/CIRCEP.117.005018.

12. Sevaptsidis E et al., “Simultaneous unipolar and bipolar recording of cardiac electrical activity”, Pacing Clin Electrophysiol., 1992, January: 15(1):45-51. https://www.ncbi.nlm.nih.gov/pubmed/1370999.

1. A multi-electrode implantable device comprising:
a lead;
a tip at a distal end of the lead;
four electrodes embedded in a tetrahedral configuration at the distal end of the lead; and
four individual wires extending from the electrodes within the lead for receiving voltages sensed by the four electrodes.

2. The multi-electrode device of claim 1, wherein a central electrode of the four electrodes is at the tip of the device, aligning a tip of the tetrahedral configuration with a longitudinal center axis of the lead.

3. The multi-electrode device of claim 2, wherein three of the four electrodes are positioned in an equilateral triangular planar configuration to form a base of the tetrahedral configuration near a circumference of the lead, the three electrodes being equally spaced in relation to the central electrode at the distal tip.

4. (canceled)

5. The multi-electrode device of claim 1, wherein the device comprises a single connector pin at a proximal end of the lead that is coupled to the wires and configured to transmit electrophysiological signals sensed by the electrodes to an external device.

6. (canceled)

7. The multi-electrode device of claim 1, wherein the device comprises a communication unit that is coupled to the four individual leads for wirelessly transmitting the sensed voltages to another device and/or the device comprises a miniaturized pacemaker/ICD unit that is coupled to the four individual leads for receiving and processing the sensed voltages.

8. (canceled)

9. A method of analyzing electrophysiological (EP) data from a 3D multi-electrode device having four electrodes positioned at a distal end of the device in a 3D tetrahedral configuration and the device being located at a heart, the method comprising:
sensing unipolar voltages with the four electrodes individually to provide sensed signals;
recording the sensed signals as the EP data after signal capture occurs;
determining an electric field from the EP data measured by the four electrodes;
generating one or more features derived from the electric field; and
analyzing the one or more features to determine when a heart rhythm of the heart has an irregular change, is erratic or is abnormal.

10. The method of claim 9, wherein the method comprises determining an electric field span potential (EFSP), a geometric profile of the electric field, a travelling wave conduction direction and/or a conduction wave velocity as the one or more features derived from the electric field.

11. The method of claim 10, wherein the method comprises determining the EFSP by determining a largest Euclidean distance formed with the electric field and scaling the Euclidean distance with an inter-electrode distance to express the EFSP as a voltage.

12. (canceled)

13. The method of claim 10, wherein the geometric profile is obtained by forming a loop when plotting 3 electric field vector components of the electric field; and/or the method comprises determining a change in the conduction wave direction by determining a change in angle of a direction axis of the electric field, where the angle is a projected angle of the electric field that is used to determine the EFSP.

14. (canceled)

15. The method of claim 10, wherein the conduction velocity is determined by taking a product of a direction vector of the electric field and a ratio of a time derivative of an average signal representing an electrode configuration over a sensed peak-to-peak bipole voltage, where the direction vector is a directional axis where a largest voltage of the electric field lies which is determined by rotation and projection of the electric field.

16.-17. (canceled)

18. The method of claim 10, wherein the method further comprises detecting changes in the electric field geometry to distinguish a change or shift in direction of a traveling wave due to different arrhythmogenic sources.

19. The method of claim 9, wherein the method comprises determining the electric field by:
taking an amplitude difference between each unique pair of unipolar signals from each electrode;
forming a spatial displacement matrix comprising the set of unique electrode pairs and their corresponding physical displacement coordinates; and
determining a negative of a product between the set of derived bipoles and the inverse of the spatial displacement matrix.

20. The method of claim 9, wherein the method further comprises:
detecting a first direction of an activated wave by aligning the activated wave with a first longest axis which is a first largest amplitude of a first electric field recorded during normal sinus rhythm;
comparing the first direction with a second direction of a second longest axis which is a second largest amplitude of a second electric field recorded during pace-mapping; and
providing a score of how similar the first direction is to the second direction.

21. The method of claim 9, wherein the method further comprises:
recording the sensed signals as the EP data after signal capture occurs during a normal cardiac rhythm;
storing a normal electric field that is derived from the recorded EP data during the normal cardiac rhythm;
defining a normal template from an electric field geometry for the stored normal electric field that is associated with the normal cardiac rhythm;
determining a matching score by taking a correlation of the normal template with electric field geometries from a later determined electric field for a given heart location;
comparing the matching score to a matching score threshold to identify when the later determined electric fields are associated with an abnormal cardiac rhythm to identify changes in heart rhythm or morphologies that are different compared to the normal cardiac rhythm; and
when the later determined electric field is abnormal, providing a pacing stimulus to induce normal cardiac rhythm.

22. The method of claim 9, wherein the method further comprises:
recording the sensed signals as the EP data after signal capture occurs during an arrhythmia;
storing an arrhythmia electric field that is derived from the recorded EP data during the arrhythmia;
defining an abnormal template from a first electric field geometry for the stored arrhythmia electric field that is associated with the arrhythmia;
determining a second electric field having a second electric field geometry resulting from pacing at a given heart location during a medical procedure with a roving ablation catheter;
determining a matching score by taking a correlation of the first electric field geometry with the second electric field geometry;
comparing the matching score to a matching score threshold to identify when the second electric field matches the arrhythmia electric field; and
when the second electric field matches the arrhythmia electric field, indicating to a medical practitioner that a remedial action be taken comprising ablation or cryofreezing at the given heart location where the pacing by the roving ablation catheter caused the second electric field.

23. The method of claim 9, wherein the method further comprises using the electric field to maximize His detection by rotating the electric field to emphasize His-Bundle activity while suppressing cardiac muscle activation.

24. (canceled)

25. A method for providing cardiac pacing using a lower stimulus threshold using a pacemaker device and a multi-electrode device located at a heart location, the device having a 3D electrode configuration as defined in claim 1, wherein the method comprises:
sensing voltages at the heart location using the electrodes;
defining combinations of an anode and cathode for each combination of the electrodes, and for each combination of the electrodes and a body of the pacemaker device;
determining sensed voltages for each of the anode and cathode combinations;
determining a pacing stimulus for each of the anode and cathode combinations using the pacemaker;
selecting the anode and cathode combination having the pacing stimulus with the lowest amplitude voltage; and
using the selected anode and cathode combination to provide pacing stimuli to the heart.

26. The method of claim 25, wherein the method is repeated periodically to determine and use the anode and cathode combination having the pacing stimulus with a lowest amplitude voltage.

27. (canceled)

28. A system for analyzing electrophysiological (EP) data from a 3D multi-electrode device having four electrodes positioned at a distal end of the device in a 3D tetrahedral configuration and the device being located at a heart, wherein the system comprises:
a data store comprising program instructions stored thereon for executing methods; and
at least one processor coupled to the data store, the at least one processor being configured to execute the program instructions to perform a method according to claim 9.

29.-43. (canceled)

44. A system for providing cardiac pacing using a lower stimulus threshold using a pacemaker device and a multi-electrode device located at a heart location, the device having a 3D tetrahedral configuration, wherein the system comprises:
a data store comprising program instructions stored thereon for executing methods; and
at least one processor coupled to the data store, the at least one processor being configured to execute the program instructions to perform a method according to claim 25.

45.-47. (canceled)
